(19) United States
(12) Patent Application Publication
(10) Pub. No.: US 2024/0008451 A1
(43) Pub. Date: Jan. 11, 2024

(54) COOLING ARTICLE AND METHOD OF USING THE SAME

(71) Applicant: Achieve Equine LLC, Delano, MN (US)

(72) Inventors: Jan WEBER, Maastricht (NL); James R. CHIAPETTA, Delano, MN (US); Hanna HARTMAN, New Germany, MN (US)

(21) Appl. No.: 18/014,928

(22) PCT Filed: Jul. 7, 2021
(86) PCT No.: PCT/US2021/040720

§ 371 Date: Jan. 6, 2023

Related U.S. Application Data
(60) Provisional application No. 63/159,200, filed on Mar. 10, 2021.
(60) Provisional application No. 63/048,981, filed on Jul. 7, 2020.

(51) Int. Cl. A01K 13/00; B68C 5/00

(52) U.S. Cl. CPC A01K 13/008; B68C 5/00; A01K 13/007

(57) ABSTRACT

A cooling article includes thermally conductive material and heat absorbing material comprising a phase change material having a phase change temperature greater than 0° C. The cooling article may be used to cool a subject or the limb of a subject. A cooling article may include passive cooling material and reinforcing fibers configured to support the passive cooling layer, where the cooling article is configured to conform to a portion of a subject's body. The cooling article may be a sheet, a blanket, or a sleeve. A method of cooling a subject includes cooling the cooling article to a temperature below the phase change temperature; and applying the cooling article to the subject.

### RELATED APPLICATIONS

This application claims priority to U.S. Provisional Patent Application No. 63/048,981 filed on Jul. 7, 2020, entitled COOLING SLEEVE AND METHOD OF USING THE SAME and to U.S. Provisional Patent Application No. 63/159,200 filed on Mar. 10, 2021, entitled COOLING ARTICLE AND METHOD OF USING THE SAME, the contents of which are incorporated herein by reference.

### FIELD

The present disclosure relates to cooling devices. In particular, the present disclosure relates to a cooling article for cooling or maintaining the temperature of at least a portion of an animal or human body, including, for example, the limb or body of a subject.

### BACKGROUND

Existing cooling and cold therapy methods include the use of cold water (e.g., running water or ice water), ice packs, and cooling products that include volatile substances that cool upon volatilization. However, drawbacks of existing methods and products include logistical challenges (e.g., use of running water or ice water), lack of control over the amount or cooling and potential damage to skin and nerves from too aggressive cooling (e.g., the use of ice packs and similar products), the cost and ecological issues experienced with single-use products (e.g., use of products that rely on volatile compounds), and the sometimes unpleasant smell of volatiles.

### SUMMARY

A cooling article includes a thermally conductive layer; and a heat absorbing layer comprising a phase change material having a phase change temperature greater than 0° C. The cooling article may be used to cool a subject, such as a human or an animal. The cooling article may be used to cool the limb of a subject. The phase change material may be encapsulated. The phase change material may be in a flowable form. The phase change material may be embedded in a thermally conductive material including a gel, a grease, a paste, a porous ceramic, a porous mineral, or a soft elastomer including potentially thermally conductive elements. The phase change material, when embedded in the thermally conductive material, may be flowable at temperatures above and below the phase change temperature. The cooling article may further include an insulating layer. The thermally conductive layer may form one or more packets encasing the phase change material. The packets may be selectively removed from the cooling article. The packets may be disposed within one or more compartments in the cooling article. The cooling article may include an inner layer constructed to be placed against the subject to be cooled. The inner layer may include a thermally conductive material. The inner layer may include a plurality of protrusions extending from an inner surface of the cooling article. The plurality of protrusions may include a thermally conductive material. The cooling article may be configured as a sleeve conformable around a limb. The sleeve may include a protective outer shell. The cooling article may be configured as blanket.

The cooling article may include a passive cooling layer. The passive cooling layer may be configured as the outer layer of the cooling article. The cooling article may further include a supportive mesh adjacent the passive cooling layer.

A heat absorbing composition may include a thermally conductive substrate; and a phase change material dispersed in the thermally conductive substrate. The thermally conductive material may include a gel, a grease, a paste, a porous ceramic, a porous mineral, or a soft elastomer.

In some embodiments, the cooling article includes a passive cooling layer having reinforcing fibers reinforcing fibers configured to support the passive cooling layer, wherein the cooling article is configured to conform to a portion of a subject's body. The reinforcing fibers may be woven into the passive cooling layer. The reinforcing fibers may be disposed adjacent the passive cooling layer.

The cooling article is configured to conform to a portion of a subject's body. The cooling article may be formed as a sleeve or a blanket. The cooling article may include additional layers and materials to impart additional properties to the cooling article. The cooling article may include a phase change material. The cooling article may include a thermally conductive substrate and a phase change material dispersed in the thermally conductive substrate.

A cooling article may include a sleeve constructed to be applied to a body of a subject, the sleeve including a dorsal region positionable adjacent a dorsal aspect of the limb; a palmar/plantar region positionable adjacent a palmar aspect of the limb; a medial region positionable adjacent a medial aspect of the limb; a lateral region positionable adjacent a lateral aspect of the limb; a heat absorbing region that is present in one or more of the palmar/plantar, medial, and lateral regions; a heat convective region that is present in the dorsal region; an inner layer forming a contact surface with the limb; an outer layer; and a phase change material disposed within the heat absorbing region between the inner layer and the outer layer. The cooling article may further include a thermally conductive layer disposed in the heat absorbing region between the inner layer and the outer layer, wherein the phase change material is dispersed in the carrier. The thermally conductive layer may include a carrier including a thermally conductive substrate; and a phase change material dispersed in the thermally conductive substrate. The outer layer may include an insulating layer adjacent the thermally conductive layer, and a passive cooling layer.

In an embodiment, the cooling article is an equine leg boot including a dorsal region positionable adjacent a dorsal aspect of the limb; a palmar/plantar region positionable adjacent a palmar/plantar aspect of the limb; a medial region positionable adjacent a medial aspect of the limb; a lateral region positionable adjacent a lateral aspect of the limb; a heat absorbing region that is present in one or more of the palmar/plantar, medial, and lateral regions. The heat absorbing region includes a phase change material having a phase change temperature. In some embodiments the heat absorbing region is compliant (e.g., malleable, conformable to the contours of the anatomy of the subject) at temperatures above and below the phase change temperature. The phase change material is disposed within a packet, where the packet may be selectively removed from the equine leg boot. The packets includes a thermally conductive layer.

A method of cooling a limb of a subject includes cooling the cooling article to a temperature below the phase change temperature; and applying the cooling article to the limb. A system for providing cooling to a limb includes a cooling sleeve; and a portable cooling device comprising a housing constructed to receive the cooling sleeve and to cool the phase change material of the cooling sleeve to below the phase change temperature.

### BRIEF DESCRIPTION OF FIGURES

FIG. 1A is schematic perspective view of a cooling sleeve according to an embodiment.

FIG. 1B is a schematic cut-off view of the wall of the cooling sleeve of FIG. 1A.

FIG. 2 is a schematic cross-sectional view of the cooling sleeve of FIG. 1A according to an embodiment.

FIG. 3A is a schematic view of a system including the cooling sleeve of FIG. 1A according to an embodiment.

FIG. 3B is a schematic view of another system including cooling packets of a cooling article according to an embodiment.

FIG. 4A is a schematic view of a cooling article according to an embodiment.

FIGS. 4B-4D are cross sectional views of the cooling article of FIG. 4A according to alternative embodiments.

FIG. 5A is a schematic view of a cooling article configured as an equine blanket according to an embodiment.

FIGS. 5B-5D are cross-sectional views of the cooling article of FIG. 5A according to alternative embodiments.

FIG. 6 is a simplified schematic of equine leg anatomy.

FIG. 7 is schematic perspective view of a cooling sleeve on a horse's leg according to an embodiment.

FIG. 8 is schematic perspective view of a cooling sleeve with a protective hard shell according to an embodiment.

FIG. 9A is schematic perspective view of a cooling sleeve on a horse's leg according to an embodiment.

FIG. 9B is a cross-sectional view of the cooling article of FIG. 9A according to an embodiment.

FIG. 10A is schematic perspective view of a cooling sleeve with a protective shield and cooling material packet according to an embodiment.

FIG. 10B is schematic perspective view of the cooling material packet of FIG. 10A according to an embodiment.

### DEFINITIONS

All scientific and technical terms used herein have meanings commonly used in the art unless otherwise specified. The definitions provided herein are to facilitate understanding of certain terms used frequently herein and are not meant to limit the scope of the present disclosure.

Unless otherwise indicated, the terms “polymer” and “polymeric material” include, but are not limited to, organic homopolymers, copolymers, such as for example, block, graft, random and alternating copolymers, terpolymers, etc., and blends and modifications thereof. Furthermore, unless otherwise specifically limited, the term “polymer” shall include all possible geometrical configurations of the material. These configurations include, but are not limited to, isotactic, syndiotactic, and atactic symmetries.

The term “elastomer” is used here to refer to a polymer with viscoelasticity (both viscosity and elasticity). Elastomers typically exhibit weak intermolecular forces, low Young's modulus, and high failure strain. The hardness of an elastomer can be measured using a durometer and expressed on a shore hardness scale. For example, a soft elastomer may be one that has a shore OO hardness value between 20 and 40, or about 30.

Unless otherwise specified, “particle size” refers to a D90 particle size (90 wt-% of the particles fall under the given particle size), measured by light scattering.

The term “thermally conductive” is used here to refer to materials that have thermal conductivity of 1 W/(m·K) or greater.

The term “subject” is used here to refer to any subject that may benefit from use of the cooling article, such as humans and non-human vertebrates. Non-human vertebrates include livestock animals, companion animals, and laboratory animals. Examples of non-human vertebrates include, without limitation, horses, camels, cows, pigs, goats, dogs, cats, and the like. In some embodiments, a subject is a mammal, particularly a horse or a human.

As used herein, the term “cooling” refers to the removal of thermal energy from the body. Accordingly, the term “cooling article” is used here to refer to an article that removes thermal energy from a subject body to either lower its temperature or to counteract an increase in temperature due to internal or external factors. A cooling article may (i) lower the temperature of all or a portion of a subject body below homeostatic temperature, (ii) maintain the temperature of all or a portion of a subject body at homeostatic temperature under conditions which would, but for the use of an embodiment of the invention, cause elevation of the temperature above the homeostatic temperature, (iii) maintain the temperature of all or a portion of the subject body above homeostatic temperature which but for the use of an embodiment of the disclosure, the temperature is below a temperature that all or the portion of the body would otherwise increase to under a particular condition; or (iv) reduce the rate of increase in temperature of all or a portion of the body which, but for the use of an embodiment of the invention, would cause the temperature of all or a portion of the body to increase at a faster rate. As an example, the “homeostatic” temperature of a horse is typically 37.5-38.5° C. Under some conditions of intensive exercise certain tissues of horse's body, such as the superficial digital flexor tendon can reach temperatures of 45° C. As used in this example, and unless otherwise stated in the disclosure, a cooling article is an article that lowers the tendon temperature below 38.5° C., maintains the temperature at 37.5-38.5° C., or maintains the temperature below 45° C. when it otherwise would have reached 45° C. or slows the rate at which the temperature would have reached 45° C., without the presence of a cooling article of the disclosure.

The terms “treating” and “treatment” can include therapeutic and/or prophylactic treatments. “Treating” and “treatment,” as used here, are not intended to be absolute terms. Treatment may lead to an improved prognosis or a reduction in the frequency or severity of symptoms. Desirable effects of treatment include preventing occurrence or recurrence of disease, alleviation of symptoms, diminishment of any direct or indirect pathological consequences of a condition, decreasing the rate of progression of a condition, amelioration or palliation of a condition, and remission or improved prognosis. Likewise, the term “preventing,” as used herein, is not intended as an absolute term. Instead, prevention refers to delay of onset, reduced frequency of symptoms, or reduced severity of symptoms associated with a condition. Prevention therefore refers to a broad range of prophylactic measures that will be understood by those in the art. In some circumstances, the frequency and severity of symptoms is reduced to non-pathological levels. In some circumstances, the symptoms of a subject upon use of the sleeve are only 90, 80, 70, 60, 50, 40, 30, 20, 10, 5 or 1% as frequent or severe as symptoms experienced by an untreated subject with the condition.

The term “substantially” as used here has the same meaning as “significantly,” and can be understood to modify the term that follows by at least about 90%, at least about 95%, or at least about 98%.

The term “not substantially” as used here has the same meaning as “not significantly,” and can be understood to have the inverse meaning of “substantially,” i.e., modifying the term that follows by not more than 25%, not more than 10%, not more than 5%, or not more than 2%.

The term “about” is used here in conjunction with numeric values to include normal variations in measurements as expected by persons skilled in the art, and is understood to have the same meaning as “approximately” and to cover a typical margin of error, such as ±5% of the stated value.

Terms such as “a,” “an,” and “the” are not intended to refer to only a singular entity, but include the general class of which a specific example may be used for illustration.

The terms “a,” “an,” and “the” are used interchangeably with the term “at least one.” The phrases “at least one of” and “comprises at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination of any two or more of the listed elements.

The recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” or “at least” a particular value, that value is included within the range.

As used here, “have”, “having”, “include”, “including”, “comprise”, “comprising” or the like are used in their open-ended sense, and generally mean “including, but not limited to.” It will be understood that “consisting essentially of,” “consisting of,” and the like are subsumed in “comprising” and the like. As used herein, “consisting essentially of,” as it relates to a composition, product, method or the like, means that the components of the composition, product, method or the like are limited to the enumerated components and any other components that do not materially affect the basic or novel characteristic(s) of the composition, product, method or the like.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure, including the claims.

Any direction referred to here, such as “top,” “bottom,” “left,” “right,” “upper,” “lower,” and other directions and orientations are described herein for clarity in reference to the figures and are not to be limiting of an actual device or system or use of the device or system. Devices or systems as described herein may be used in a number of directions and orientations.

### DETAILED DESCRIPTION

The present disclosure relates to cooling articles, devices, and systems. In certain embodiments, the present disclosure relates to full body cooling systems such as a body blanket, a cooling sleeve for localized cooling of an appendage or a joint, or other configurations of articles for localized cooling of a part of the body.

Cooling of the body or a region of the body may be desirable for both human and animal subjects. For example, lowering the homeostatic temperature of the body or region of the body, maintaining the temperature, lowering the temperature below homeostatic or slowing the rate of increase of the temperature of a tissue may be desirable under different circumstances. Use of an embodiment of the invention may include cooling before, during, or after an athletic performance, or treating an injury or illness.

Heat transfer from living bodies that supports cooling may occur in multiple ways. Convection cools a body through evaporation of sweat expelled through pores. Optimal efficacy is dependent on replacement of humidified air adjacent the body with fresh air. Conduction cools a body by transferring heat directly from a warm body to a cool body. The amount of heat removed by conduction is controlled by temperature difference between the bodies, heat conductance resistance, and the total contact area between hot and cold bodies. Conventional cooling articles (e.g., ice packs) typically rely on conduction. Radiation, as the term implies, cools the body by heat radiating away from the body. Radiation is controlled by the temperature of the body, although radiation may be blocked or countered by radiation back to the body by the environment. In mass heat transport, a warmer substance is replaced by a cooler substance, transferring heat away. Examples of mass heat transport are cooling by wind or moving water, or tissues being cooled by cooling the circulating blood perfusing the tissue.

In situations it may be desirable to provide cooling to a localized region of the body such as a limb of a subject. In some body regions, anatomical and physiological characteristics within the region may be cooled effectively by unique and novel configurations, combinations and/or arrangements of cooling materials having properties that are different than the arrangement and/or configuration of cooling materials at another location within the same region. In embodiments, the arrangement and/or configuration of one, two or more of the of cooling materials at a particular location of a cooling system for that region may be the same or different than that at another location within the region depending on the anatomy or physiology of the region of the body to be cooled.

According to an embodiment, cooling is provided by the use of a cooling article positioned on the body. For example, cooling the body (e.g., cooling of the torso) may be achieved by providing a cooling “blanket” that covers some or at least a part of the torso. In examples of this embodiment, the cooling blanket may be a flat sheet that drapes the body with particular openings, contours, and configurations. Such openings, contours, and configurations are known in the art of human, equine, or canine blankets. Suitable fasteners for maintaining position of the article are known in the art and are suitable for use according to the invention.

In embodiments, for cooling an appendage, the cooling article can be configured to conform to the particular region of the body using openings, contours, configurations and fasteners known in the art.

In embodiments, the cooling article includes a heat absorbing material that is conformable below the phase change temperature of the heat absorbing material (e.g., the phase change material). For example, the heat absorbing material may be contained in a carrier forming a packet, where the packet remains pliable and may be conformed to the shape of a body part (e.g., a limb) even when cooled to below the phase change temperature. The term “pliable” is used here to refer to a material that is readily conformable to the anatomy of a subject. For example, a block of ice would not be considered “pliable” but a bag of sand or wet clay would be.

According to embodiments, the cooling article includes one or more cooling materials in arrangements and configurations that enable different cooling mechanisms or function more efficiently. Cooling materials may include heat absorbing materials, passive cooling materials, thermally conductive materials, insulating materials, and configurations, structures and arrangements that facilitate heat transfer. The cooling property of a material may be due to the chemical composition, structure, or both chemical composition and structure. The cooling benefits of the cooling article may further be affected by the arrangement of the materials relative to one another and relative to the local physiology or anatomy of the region the body.

In some embodiments, a cooling article includes arrangements and configurations to enable two or more cooling mechanisms including convection, conduction, radiation, and mass heat transport. Accordingly, the cooling article may include one or more features that draw heat away from a surface of the subject, features that conduct heat, features that absorb heat, features that reduce or prevent further heating by reflecting external sources of heat, or features that facilitate losing heat by radiation from the body while preventing incoming radiation from external sources.

In some embodiments, the cooling article includes one or more materials that absorb heat, and optionally one or more additional features that transfers heat away from a surface of the subject, that conduct heat, or that reduce or prevent further heating by reflecting external sources of heat.

The different cooling mechanisms may also provide cooling that is differentiated in time (e.g., one cooling mechanism provides initial cooling and another cooling mechanism provides subsequent cooling) or place (e.g., one cooling mechanism provides cooling of one area of the subject or region and another cooling mechanism provides cooling of another area).

The cooling article may further include aspects that eliminate barriers to cooling such as isolation or insulation. The cooling article may include one or more features that draw heat away from a surface of the subject, features that conduct heat, features that absorb heat, or features that reduce or prevent further heating by reflecting external sources of heat. In some embodiments, the cooling article includes two or more, or three or more of such features. According to an embodiment, the cooling article includes one or more materials that absorb heat (e.g., heat absorbing materials), and optionally one or more additional features that draw heat away from a surface of the subject, that conduct heat, or that reduce or prevent further heating by reflecting external sources of heat.

The cooling article may provide other functions and benefits in addition to cooling, such as protection from injuries (e.g., blunt force trauma) or protection from the environment (e.g., heat, sunlight, etc.) including insects, dust, etc.

Passive Cooling Materials

In an embodiment a cooling material can advantageously provide passive cooling. Such passive cooling materials may be used alone or in configurations and arrangements disclosed herein for a particular cooling article. Some passive cooling materials described herein can support cooling by allowing infrared radiation from a warm body or appendage to escape away from the body to the outer atmosphere while sunlight (visible, UV, and near infrared spectrum), often present during outdoor exercise, is reflected.

Radiation is reflected by structures having dimensions similar to the wavelength of the radiation. As such, sun light with main wavelength components in the range 100-1000 nm, is strongly reflected by materials having structural dimensions in this order of magnitude. One example being polymer fibers or sheets with nano porosity having dimensions in this 100-1000 nm range.

Suitable passive cooling fabrics include nanoporous polymeric fabrics. Examples of such nanoporous polymeric fabrics include nanoporous polyethylene described by Hsu et al. in Radiative Human Body Cooling by Nanoporous Polyethylene Textile, 353 Science 1019 1020 (2016), and by Yu et al. in Thermoconductive, Moisture-Permeable, and Superhydrophobic Nanofibrous Membranes with Interpenetrated Boron Nitride Network for Persona Cooling Fabrics, 12 ACS Applied Mater. & Interfaces 32078 (2020). Other examples of suitable passive cooling fabrics are a two-layer composite of nanofiber polyamide-polyethylene, described by Song et al. in Wearable Polyethylene/Polyamide Composite Fabric for Passive Human Body Cooling, 10 ACS Applied Mater. & Interfaces 41637 (2018), and a three-layer composite of nanofiber polyamide-polyvinylidene fluoride-polyethylene, described by Song et al. in Novel Passive Cooling Composite Textile for Both Outdoor and Indoor Personal Thermal Management, 130 Composites A 105738 (2020), or Lili Cai et al., Spectrally Selective Nanocomposite Textile for Outdoor Personal Cooling, Adv. Mater. 1802152 (2018). Suitable nanoporous polymeric fabrics may have a porosity with dimensions that correspond to the wavelength of sun light, in the range of 100 nm to 1000 nm. An alternative approach to nano-porosity to create specific reflection is to include nanoparticles with similar dimensions in the range of 100 nm to 1000 nm. As explained by Lili Cai, embedding zinc oxide nanoparticles (NPs) into nanoporous polyethylene (ZnO-PE) offers a cooling power of about 200 W/m², compared to conventional textiles like cotton under typical outdoor environment with peak solar irradiance of over 900 W/m². The principle is the same as for the fibers—embedding structures, in this case ZnO nanoparticles, with dimensions in the range of the solar wavelengths (100 nm to 1000 nm) to achieve high reflection in visible and near-IR region and high transmission in mid-IR region where the body is emitting heat radiation.

The passive cooling fabrics block the dominant frequency range of the thermal radiation from the sun while allowing the thermal radiation of the body to pass. The principle is based on a scattering theory which states that a material can strongly scatter electromagnetic waves with a wavelength close to the material's size dimensions. Solar spectrum is mainly composed of visible light (400-700 nm) and near IR (NIR) light (700-2,500 nm), which together constitute around 93.4% of solar irradiance (1,000 W/m²). The body's thermal radiation occurs at much longer wavelengths, between 8 and 13 μm, peaking at 9.5 μm. According to an embodiment, the cooling article includes a passive cooling fabric that acts as a reflector (greater than 80% reflectivity) for frequencies from 300 nm up to 2500 nm while being transparent (greater than 80% transparency) for longer wavelengths.

Depending on the materials of individual layers, the fabric may be hydrophilic or hydrophobic.

Due to the nanoscale fibers used in the nanoporous polymeric fabrics, the fibers may be somewhat fragile. It may be desirable to add a supportive structure to the passive cooling material. Suitable supportive structures include materials that provide sufficient support for the nanofibers but do not interfere with the passive cooling function. In some embodiments, the passive cooling material comprises an additional layer of a cooling article. The additional layer may include a supportive open mesh. The supportive mesh may be included on one side (e.g., the outer side) or on both sides of the passive cooling material. In some embodiments, the cooling article includes a supportive mesh as an outer layer. The supportive mesh may be made from any suitable material, such as a polymer (e.g., polyethylene, polypropylene, polyamide, polyester, or the like), or a metal, or an organic fiber such as cellulose. The mesh size (the size of the openings) of the supportive mesh may be 2 mm or greater, 3 mm or greater, 4 mm or greater, or mm or greater. The mesh size may be 15 mm or less, 12 mm or less, or 10 mm or less. In some embodiments, the mesh size is from 3 mm to 10 mm.

In one embodiment, the supportive mesh is made from a high density polyethylene, such as the ultra-high molecular weight polyethylene (UHMwPE) commercially available as DYNEEMA® from Koninklijke DSM N. V. in Heerlen, the Netherlands. The benefit of an ultra-high molecular weight PE is that the fiber is light but strong and can offer support without adding much weight and without substantially interfering with the passive cooling of the nanoporous polymeric fabric. The support mesh may be attached to the nanoporous polymeric fabric by any suitable technique, such as sewing or by an adhesive. The layers may further be quilted together using a pattern that allows for some elongation of the fabric.

In embodiments, such passive cooling materials, with or without a supporting mesh, can be arranged as a component in the configuration of a blanket or sheet to cover some or all of a body of a subject. In embodiments, a passive cooling material with or without a supportive mesh can be configured and arranged with other materials disclosed herein to form a cooling article of the invention.

In some embodiments the supportive structure includes reinforcing fibers that are dispersed throughout the herein disclosed fabrics such as by being weaved within the fabric or by being incorporated in a multilayer construction in which one or more of the layers are made in part or entirely out of reinforcement fibers. The layers can be attached to each other for example using glue or stitches.

Spacers

The cooling article may further include spacers that provide space between the cooling article (e.g., blanket, sheet, or sleeve) and the body and that facilitate airflow between the cooling article and body to increase the opportunity for convection cooling. The spacers may be provided on a portion of a blanket or a cooling sleeve. The spacers may be provided along the entire inner surface of a blanket or a cooling sleeve.

The spacers may be provided as extensions protruding from an inner surface of the article or as an integral part of the surface of the article. For example, spacers may be formed by additive manufacturing, including extrusion and 3D-printing. Spacers may be directly extruded or 3D-printed onto the surface of material (e.g., onto the surface of fabric) used to form at least a portion of the inner surface of the cooling article. The spacers may be formed separately and adhered to the fabric. The spacers may be formed by a patterned, pre-formed three dimensional structure (e.g., pleats, bumps, or the like) of the inner surface of the cooling article itself.

The spacers may be provided in a regular or non-regular pattern. The spacing or density of spacers may be varied throughout the cooling article's inner surface. For example, in a cooling blanket, spacers may be more densely applied in areas that experience higher external pressure due to external compression forces (e.g., buckles or fasteners) or the weight of the cooling article itself. In a cooling blanket for a horse, spacers may be more densely applied in the area of the blanket that covers the center back of the horse and may be between a saddle and the horse. The spacers may be positioned to optimize airflow between the spacers and from edges of the cooling article to a center region of the cooling article.

In an embodiment, the spacers are 3D-printed structures extending from an inner surface of the cooling article. The spacers may be 3D-printed structures extending from an inner surface of the cooling article in a regular pattern. The spacers may have any suitable shape, such as a simple protrusion extending from the surface, or a U-shaped loop forming a loop pile. In embodiments, the spacers can be applied using known extrusion techniques. Suitable materials for spacers can include any non-irritating materials that resist the amount of external compression force desired such as wood, polymers (e.g., polyurethane, nylon, polyethylene, polypropylene, rubber, silicone, etc.), metal, etc.

The size and compression resistance of the spacers may vary throughout the cooling article. In some areas, where more airflow or reduced compression to allow continued airflow are desired, the spacers may be larger and/or have increased resistance to compression. In some embodiments, where the spacers are provided by a patterned, pre-formed three dimensional structure, the weaving pattern or weave density may vary to provide areas of higher or lower resistance to compression.

Heat Absorbing Materials

According to an embodiment, the cooling article may include a heat absorbing material. Preferably, the heat absorbing material provides a cooling capacity that provides controlled and efficient cooling without damage to skin or other tissues.

In some embodiments, the heat absorbing material includes a phase change material (“PCM”). A PCM is a material that is capable of absorbing or releasing thermal energy (e.g., heat) due to a phase change at the phase change temperature of the material. Phase changes may include change from a liquid to a solid (e.g., solidification or freezing), solid to a liquid (e.g., melting), amorphous (or liquid) to a crystal (e.g., crystallization), or crystal to amorphous. According to an embodiment, the cooling article includes one or more PCMs.

The PCM may be selected to have a phase change temperature in a selected temperature range for particular cooling article. For example, the phase change temperature may be −15° C. or greater, −10° C. or greater, −5° C. or greater, 0° C. or greater, 2° C. or greater, 4° C. or greater, 6° C. or greater, 8° C. or greater, 10° C. or greater, 15° C. or greater, or 20° C. or greater. The phase change temperature may be 30° C. or lower, 20° C. or lower, 15° C. or lower, 10° C. or lower, 5° C. or lower, or 2° C. or lower. In some embodiments, the phase change temperature is in the range of −15° C. to 30° C., −5° C. to 20° C., 0° C. to 15° C., 5° C. to 20° C., or from 1° C. to 8° C.

The cooling article may include two or more PCMs having different phase change temperatures. For example, the heat absorbing layer may include 2, 3, 4, 5, 6, 7, 8, 9, or even 10 different PCMs. At least some or each of the PCMs have a different phase change temperature. The PCMs may be selected so that a first PCM provides initial fast cooling to drop the temperature quickly, and a second PCM provides cooling at a higher phase change temperature than the first PCM, which provides a slower rate of cooling. The phase change temperature of the second PCM may be higher than the phase change temperature of the first PCM but lower than the subject's (e.g., human's or animal's, e.g., horse's) normal ambient temperature.

In one embodiment, the heat absorbing layer includes a first PCM with a phase change temperature in a range of −12° C. to 5° C., from −10 to 5° C., or from −10° C. to 0° C. The heat absorbing layer includes a second PCM having a phase change temperature greater than the first PCM. The phase change temperature of the second PCM may be in a range of 0° C. to 20° C., from 5° C. to 15° C., from 2° C. to 10° C., or from 4° C. to 8° C. The heat absorbing layer may optionally include a third PCM having a phase change temperature greater than the second PCM. The phase change temperature of the third PCM may be up to 20° C., or in a range of 4° C. to 30° C., from 6° C. to 20° C., or from 8° C. to 18° C. In embodiments, more than three PCM materials may be incorporated to provide incrementally increasing phase change temperatures such as a first PCM with a phase change temperature of 0° C., a second PCM with a phase change temperature of 8° C., a third PCM with a phase change temperature of 15° C., and a fourth PCM with a phase change temperature of 18° C.

The PCM may be made from a vegetable-based material, petroleum-based material, water-based material, mineral-based material, or a combination thereof. In some embodiments, the PCM includes a vegetable-based material. Examples of suitable vegetable-based materials include hydrogenated fats or oils, such as, without limitation, palm oil, palm kernel oil, rapeseed oil, coconut oil, soybean oil, and combinations thereof. Vegetable-based PCMs may beneficially be non-toxic and biodegradable.

The cooling article may include a suitable amount of PCM. The amount of PCM may be selected based on the intended use and the size of the cooling article. For example, a cooling blanket intended for use on a large animal, such as a horse, may include a relatively large amount of PCM, whereas a cooling sleeve intended for use on a human wrist may include a relatively small amount of PCM. According to an embodiment, without limiting suitable ranges, the cooling article includes exemplary amounts of 10 g or more, 25 g or more, 50 g or more, 100 g or more, 150 g or more, 200 g or more, or 250 g or more of the PCM. The cooling article may include 800 g or less, 600 g or less, 400 g or less, or 250 g or less of the PCM. The amount of PCM may be determined based on the surface area of the cooling article. The surface area here is understood to mean a major surface of the article (for example, a major surface of a blanket) that may come into contact with the skin of the subject. In some embodiments, the cooling article includes 800 g/m² or less, 600 g/m² or less, 500 g/m² or less, 1000 g/m² or less, 2000 g/m², or 5000 g/m² or less of PCM. The cooling article may include 200 g/m² or more, 400 g/m² or more, 600 g/m² or more, or 1000 g/m² or more of PCM. The cooling article may include from 200 g/m² to 800 g/m² of PCM, from 200 g/m² to 600 g/m² of PCM, from 200 g/m² to 500 g/m² of PCM, from 400 g/m² to 800 g/m² of PCM, from 400 g/m² to 600 g/m² of PCM, or from 400 g/m² to 500 g/m² of PCM. The amount of PCM per surface area may be determined as an average amount across the surface area.

The PCM may be provided in any suitable form. Conveniently, the PCM may be encapsulated. For example, the PCM may be microencapsulated (e.g., encapsulated particles having a size in the range of 10 μm to 1000 μm) or macroencapsulated (e.g., encapsulated particles having a size in the range of 1 mm or greater). The PCM may be fully encapsulated or partially encapsulated. The term fully encapsulated is used to refer to an arrangement where the PCM is surrounded by an encapsulating material on all sides. The term partially encapsulated is used to refer to an arrangement where the PCM is partially surrounded by an encapsulating material and at least some of the PCM is exposed to the environment. Encapsulated PCM may include both fully encapsulated and partially encapsulated PCM. In one embodiment, the heat absorbing layer includes microencapsulated PCM. In one embodiment, the heat absorbing layer includes macroencapsulated PCM. Encapsulated PCMs may include a solid shell made of a material that does not participate in the phase change (e.g., melting) at the phase change temperature. In some cases, the encapsulation material may include a plastic (e.g., polymer). Suitable encapsulated PCMs include PCMs available from Puretemp LLC in Minneapolis, MN; BIOPCM Q5 available from Phase Change Solutions in Greensboro, NC; and CRODATHERM™ from Croda International Plc in East Yorkshire, United Kingdom. Other examples of suitable PCMs include alkyl hydrocarbons such as those disclosed in U.S. patent application Pub. No. 2003/0026973, the entire disclosure of which is incorporated herein.

The PCM may also be incorporated in a non-phase-change porous material, where the PCM is located within the pores of the porous material. Examples of such porous materials containing PCM include porous ceramics and porous minerals, such as Rubitherm® GR, a porous silicate, such as Rubitherm® PX, both available from Rubitherm Technologies GmbH in Berlin, Germany, or a nanostructured calcium silicate (NCS), such as those disclosed in Johnston, J. H., Nanostructured Calcium Silicate Phase Change Materials for Thermal Buffering in Packaging Applications, Mater. for Energy, Efficiency and Sustainability: TechConnect Briefs (2018). Other suitable porous materials may include boron nitride, aluminum oxide (Al₂O₃), aluminum nitride (AlN), aluminosilicate (Al₂SiO₅), beryllium oxide, silicon carbide, silicon nitride, yttrium oxide, calcium carbonate, carbon, etc., and combinations thereof. The porosity of the encompassing material (e.g., ceramic or mineral) can range from 20% porosity to 95% porosity. The porous material may have a particle size ranging from 1 μm to 5 mm, or from 1 μm to 5 μm (NCS), up to about 200 μm (e.g., Rubitherm PX), or from 1 mm to 3 mm (e.g., Rubitherm GR). Different sizes of particles may be mixed and included in the same packet to increase packing density.

The amount of the PCM within the encapsulated PCM may be varied to increase or decrease the relative amount of PCM relative to the encapsulating material. For example, the amount of PCM in the encapsulated PCM may be 10 wt-% or greater, 25 wt-% or greater, 50 wt-% or greater, or 75 wt-% or greater. The balance may be the encapsulating material. The amount of PCM material in the encapsulated PCM may be up to 98 wt-%.

Ideally, the heat absorbing material would be in direct contact with the skin of the subject. However, because many mammals have hair covering some or all of their body, direct contact of the skin with the heat absorbing material is not feasible. When the subject or a region of the body is covered by hair or fur, the hair or fur traps air and reduces efficiency of heat transfer from the skin to the heat absorbing material. The insulation effect may be exacerbated by a mechanism called piloerection, where hair erector muscles cause hairs to stand up to trap more air between hairs. Thus, in embodiments one or more components may be included to facilitate heat transfer from the skin to the heat absorbing material.

In addition, in the case of PCMs that are microencapsulated, dead air spaces can exist between the spherical contours of the microencapsulated particles. In these and other situations, thermally conductive materials can be used to improve efficiency of cooling including cooling articles of the disclosure.

Thermally Conductive Materials

The cooling article may include components that facilitate transfer of the heat from the subject to the heat absorbing material (e.g., the PCM). Such features may include the use of thermally conductive materials having different physical properties that are provided in various parts of the cooling article. Such features may include the use of thermally conductive layers or carriers that improve transfer of heat from the subject to the heat absorbing material. In some cases, the thermally conductive material may be provided as a layer of a multi-layer cooling article, for example, the inner layer of the article. In some cases, the thermally conductive material may be provided as a flowable material that is distributed within the heat absorbing material layer as further described below.

In embodiments, a thermally conductive material in the form of a solid material can be positioned in direct contact with the subject to facilitate the transfer of heat from the subject to the heat absorbing layer. Any suitable thermally conductive material may be used. Examples of suitable materials include metals, materials that contain metal, and material compositions that contain other thermally conductive materials, such as polymers containing thermally conducting fillers.

In one embodiment, the thermally conductive material includes stainless steel. Beneficially, stainless steel is durable, resistant to corrosion, and washable. To accommodate conforming a cooling article to the body (e.g., a cooling sleeve to a limb), the thermally conductive material may form a layer of a cooling article, for example an “inner layer.” As used herein, the “inner layer” is the layer of a cooling article located closest to the subject. When configured as a layer, the thermally conductive material can have a woven or knitted structure. In one embodiment, the thermally conductive layer includes a knitted or woven stainless steel. Examples of such material include BEKINOX® available from N. V. Bekaert S. A. in Zwevegem, Belgium. Suitable thermally conductive materials also include graphene, carbon nanotube polymers, metals (e.g., copper, silver), metals combined with diamond-like coatings (“DLC”) such as DLC coated copper wire, thermally conductive polymers such as COOLPOLY® available from Celanese Corporation in Irving, TX, metalized textile fibers (including, for example, silver or copper), such as those available from Statex Produktions and Vertriebs GmbH in Bremen, Germany (e.g., SHIELDEX® metalized fabrics), and Hengtong Chemical Group Ltd in Jiyuan, China, etc. The thermally conductive material may be a continuous layer along the article or positioned at selected locations. For example, if greater heat conductivity is desired at the proximal end or mid portion of the article only that region can include the thermally conductive layer. The thermally conductive materials in the form of textiles may be used to form packets to enclose and contain a heat absorbing material (e.g., PCM).

The inner layer of a cooling article further includes a “contact surface” which is a region of the inner layer that directly contacts the subject. In some embodiments, the contact surface includes materials, structures or textures that increase the surface area of the thermally conductive material to facilitate heat transfer from the body to the heat absorbing material. Contact surface textures can include fibers, loops, protuberances, ridges, or other protrusions to penetrate the hair or fur (e.g., in a hair-brush like manner) to increase contact surface area of the thermally conductive material with the skin.

In some embodiments the contact surface structure is constructed of a metal or other thermally conductive material. Suitable metals include metals and metal alloys that are thermally conductive and preferably also corrosion resistant and flexible. Examples of suitable metals include stainless steel, aluminum, aluminum alloy 3003, Al—Ni—La alloy (discussed in Akopyan et al., New in-situ Al matrix composites based on Al-Ni-La eutectic, 245 Materials Letters, 110-113 (2019)), copper alloys, and tinned copper filament.

In some embodiments, the protrusions are fibers that create the contact surface of the inner layer in the form of a velvet-like structure, referred to as a thermal velvet. The fibers may be made of a thermally conductive material, such as carbon or a composite of a polymer and a thermally conductive filler. The fibers may have a thickness (e.g., diameter) ranging from 1 μm to 200 μm, or from 4 μm to 12 μm. The fibers may have a length ranging from 0.5 mm to 5 mm or from 0.8 mm to 3 mm. In one embodiment, the fibers have a thickness of about 50 μm and a length of about 4 mm. The fibers may be attached to a substrate (e.g., sheet) made from any suitable material, such as metal foil, polymer, filled polymer, carbon, or the like. Preferably, the fibers are attached to a the thermally conductive material substrate. The fibers may be attached using an adhesive, such as a silicone, an epoxy, a ceramic adhesive, and the like. The fibers may be attached to the substrate at a suitable packing fraction, such as from 0.1% to 25%, or from 2% to 15%. To calculate the packing fraction, the relative density of the composite material, the casting material (e.g., epoxy), and the fiber are determined. For example, a brush layer may be cast in epoxy, thereby making in essence a composite of epoxy with a fiber reinforcement. The volume and mass are measured to calculate the relative density pC of the composite. A block of pure epoxy is cast, and the relative density pE of the epoxy is calculated. The same measurement is also performed on a large number of fibers to determine the relative density of the fiber material, pF. The packing fraction can be calculated as Absolute(pE-pC)/Absolute(pF-pE). This method is independent from the individual fiber dimensions.

Thermally conductive materials used in the cooling article also include thermally conductive polymers. The thermally conductive polymers may either be used as part of the contact surface or other parts of the cooling article. Such polymers include, for example, polymers filled with a thermally conductive filler. Suitable polymers include, for example, polypropylene, polyethylene, polyurethane, silicone-rubber, and the like. Suitable thermally conductive fillers include, for example, ceramic materials such as boron nitride, aluminum oxide (Al₂O₃), aluminum nitride (AlN), aluminosilicate (Al₂SiO₅), beryllium oxide, silicon carbide, silicon nitride; yttrium oxide, calcium carbonate, graphene, carbon, electroactive polymers (e.g., polyaniline), and combinations thereof. The filler may be provided as a nanoparticle or nanotube, such as boron nitride nanoparticles, polyaniline nanoparticles, or carbon nanotubes.

In an embodiment, the cooling article includes boron nitride. The boron nitride may be included as a thermally conductive filler in a thermally conductive polymer. Hexagonal boron nitride, also sometimes referred to as “white graphite,” is structurally similar to graphite. The boron nitride may be provided as a nanoparticle. The boron nitride may include platelets, flakes, agglomerates, or combinations thereof. By selecting a suitable particle configuration, the material may be designed to exhibit primarily heat dissipating (spreading) properties or heat transfer properties. In one embodiment, the material including boron nitride includes thermally conductive features designed to exhibit heat transfer properties. Suitable boron nitride materials are commercially available from 3M Company in St. Paul, MN, as 3M™ Boron Nitride Cooling Filler, and from Showa Denko K. K. in Tokyo, Japan as SHOBN™. Other suitable thermally conductive filler materials include alumina fillers available from Showa Denko and nano-diamonds available from Carbodeon Ltd. Oy in Vantaa, Finland.

The thermally conductive material may include a polymer filled with a thermally conductive filler. The filler loading of the filled polymer may be 10 wt-% or greater, 20 wt-% or greater, 30 wt-% or greater, or 50 wt-% or greater. The filler loading of the filled polymer may be 80 wt-% or less, 75 wt-% or less, or 70 wt-% or less. In some embodiments, the filler loading of the filled polymer is from 30 wt-% to 75 wt-%.

A suitable particle size of the filler is any size that improves heat transfer and/or heat conductance, for example, a range from 10 nm to 500 μm, or from 500 nm to 200 μm.

Contact surface features to increase surface area can include protrusions. The protrusions may be shaped as ridges providing the contact surface of an inner layer of a cooling article. The protrusions may include fibers or wires arranged as loops extending from the inner layer. The protrusions may include fibers connected to the inner layer at one end and having a free end extending from the contact surface. The protrusions may be made of the same material as the material of the inner layer, or from a different material. The protrusions may be formed of a suitable thermally conductive material, such as metal or a thermally conductive filled polymer. Suitable metals and thermally conductive filled polymers are discussed above. According to an embodiment, whether made from the same or different materials, both the protrusions of the contact surface and the remainder of the inner layer are made from a thermally conductive material.

In some embodiments, the protrusions are shaped as ridges extending from the inner surface. The ridges may have a length and a width extending along the contact surface of the cooling article (e.g., along the inner surface), and a height extending orthogonal to the surface. The length of the ridges may be 1 mm or greater, 2 mm or greater, 3 mm or greater, 4 mm or greater, or 5 mm or greater. The length of the ridges may be 20 mm or less, 15 mm or less, 12 mm or less, 10 mm or less, or 8 mm or less. In one embodiment, the length of the ridges is from 4 mm to 8 mm, or about 6 mm. The width of the ridges may be 0.5 mm or greater, 0.8 mm or greater, 1 mm or greater, 1.5 mm or greater, or 2 mm or greater. The width of the ridges may be mm or less, 4 mm or less, or 3 mm or less. In one embodiment, the width of the ridges is from mm to 3 mm, or about 2 mm. The height of the ridges may be 0.5 mm or greater, 0.8 mm or greater, 1 mm or greater, 1.5 mm or greater, or 2 mm or greater. The height of the ridges may be mm or less, 4 mm or less, or 3 mm or less. In one embodiment, the height of the ridges is from 0.8 mm to 3 mm, or about 2 mm. The ridges may be set apart from one another by a distance of 1 mm or greater, 2 mm or greater, 3 mm or greater, 4 mm or greater, or 5 mm or greater. The ridges may be set apart from one another by a distance of 20 mm or less, 15 mm or less, 12 mm or less, 10 mm or less, or 8 mm or less. In one embodiment, the ridges are set apart from one another by a distance of 4 mm to 8 mm, or about 6 mm.

In some embodiments, the protrusions include fibers or wires arranged as loops extending from the inner surface. The loops may form a looped pile on the inner surface of the cooling article. The loops may be formed of a suitable thermally conductive material, such as metal or a thermally conductive filled polymer. Suitable metals and thermally conductive filled polymers are discussed above. The loops may have any suitable shape and size. The loops may have a width extending along the surface of the cooling article (e.g., along the inner surface), and a height extending orthogonal to the surface. The fibers or wires used to make the loop may have a thickness (e.g., loop thickness). The width of the loops may be 1 mm or greater, 2 mm or greater, 3 mm or greater, 4 mm or greater, or 5 mm or greater. The width of the loops may be 12 mm or less, 10 mm or less, or 8 mm or less. In one embodiment, the width of the loops is from 2 mm to 8 mm, or about 6 mm. The height of the loops may be 0.5 mm or greater, 0.8 mm or greater, 1 mm or greater, 1.5 mm or greater, or 2 mm or greater. The height of the loops may be 12 mm or less, 8 mm or less, 5 mm or less, 4 mm or less, or 3 mm or less. In one embodiment, the height of the loops is from 2 mm to 12 mm, or about 8 mm. The loops may be set apart from one another by a distance of 1 mm or greater, 2 mm or greater, 3 mm or greater, 4 mm or greater, or 5 mm or greater. The loops may be set apart from one another by a distance of 20 mm or less, 15 mm or less, 12 mm or less, 10 mm or less, or 8 mm or less. In one embodiment, the loops are set apart from one another by a distance of 4 mm to 8 mm, or about 6 mm. The loop thickness may be 0.5 mm or greater, 0.8 mm or greater, 1 mm or greater, or 1.5 mm or greater. The loop thickness may be 3 mm or less, 2.5 mm or less, or 2.2 mm or less. In one embodiment, the loop thickness is from 1 mm to 3 mm or about 2 mm.

In some cases, the thermally conductive material may be provided as a layer of a multi-layer cooling article, for example, the inner layer of the article. In some embodiments, the thermally conductive material is used to surround a heat absorbing material such as a packet of PCM. For example, the cooling article may include an inner layer including a contact surface having features (e.g., fibers, loops, or ridges) that facilitate heat transfer from the skin to the remainder of the inner layer and the inner layer is configured to encase packets of heat absorbing materials forming the heat absorbing layer. Such features may include one or more layers of thermally conductive materials between the inner layer and the heat absorbing layer.

The thermally conductive material may form one or more packets, or cells that encase the PCM. For example, the thermally conductive material may form a quilted structure forming packets that include the PCM. In embodiments a portion of the thermally conductive material can provide an exterior surface of a packet containing the PCM which also is the contact surface of the cooling article that contacts the body. In embodiments these packets can be selectively inserted and removed from the cooling article to be cooled to below the phase change temperature before use eliminating the need to cool the entire article before use. In embodiments, rather than the exterior surface of the packet of PCM forming the contact surface of the cooling article, a layer of a thermally conductive material can be present to form the contact surface and the exterior surface of the PCM packet, which may or may not be enclosed by a thermally conductive material can be adjacent the thermally conductive material that forms the contact surface.

The thermally conductive material used to form packets of PCM can be thermally conductive materials as described above including, without limitation, a woven or knitted structure such as knitted or woven stainless steel (e.g., BEKINOX® available from N. V. Bekaert S. A. in Zwevegem, Belgium), thermally conductive polymers such as COOLPOLY® available from Celanese Corporation in Irving, TX, metalized textile fibers (including, for example, silver or copper), such as those available from Statex Produktions- and Vertriebs GmbH in Bremen, Germany (e.g., SHIELDEX® metalized fabrics), and Hengtong Chemical Group Ltd in Jiyuan, China, etc.

In some cases, a thermally conductive material may be provided as a flowable material that is distributed within the heat absorbing material layer as further described below. In some embodiments, the thermally conductive material is in the form of particles that are contained within the packets or cells and where the PCM material are dispersed within the thermally conductive material particles.

As described above, the heat absorbing material may be a PCM. The PCM may be in the shape of particles (e.g., encapsulated particles). The particles of PCM may be further embedded in a carrier material that conducts heat to the particles of PCM. The particles of PCM may be embedded in a thermally conductive substrate. In some cases, the PCM particles may be suspended in an aqueous carrier. A potential disadvantage of an aqueous carrier is that as the PCM particles are cooled below 0° C., the carrier material freezes into a solid form that is not suitably malleable for conforming a cooling article to the shape of a subject body or an appendage. According to an embodiment, the PCM may be embedded in a carrier that remains pliable at temperatures below the PCM phase change temperature and also provides thermal conductivity between the subject and the PCM. According to an embodiment, the PCM is embedded in a carrier that remains flowable at temperatures below the PCM phase change temperature and also provide thermal conductivity between the subject and the PCM. According to an embodiment, the PCM is embedded in a carrier that comprises particles that remain flowable at temperatures below the PCM phase change temperature and also provide thermal conductivity between the subject and the PCM. As used herein, such “fluid” thermally conductive materials are referred to as thermally conductive PCM carriers. Suitable thermally conductive PCM carriers include thermally conductive substrates such as gels, greases, pastes, soft elastomers, particles, and other herein disclosed soft and pliable materials, with suitable thermal conductivity. A suitable thermal conductivity may be a thermal conductivity of 2 W/(m·K) or greater, 5 W/(m·K) or greater, 10 W/(m·K) or greater, 15 W/(m·K) or greater, or 20 W/(m·K) or greater. Although there is no desired upper limit for thermal conductivity, in practice, the thermal conductivity of the material may be up to 200 W/(m·K) or up to 100 W/(m·K).

The amount of PCM particles in the thermally conductive PCM carrier may be 20 wt-% or more, 30 wt-% or more, or 40 wt-% or more. The amount of PCM particles in the thermally conductive PCM carrier may be up to 95 wt-%, 90 wt-%, 80 wt-%, or 70 wt-%.

According to an embodiment, the thermally conductive PCM carrier does not exhibit a phase change (e.g., does not change to a solid) at temperatures of −10° C. or greater or −5° C. or greater. The thermally conductive PCM carrier may be selected to have a suitable viscosity at temperatures of −10° C. or greater or −5° C. or greater The viscosity of the thermally conductive PCM carrier in the temperature range of −10° C. to 40° C. or −5° C. to 40° C. or 0° C. to 40° C. may be 25 cP (25 mPa·s) or greater or 50 cP (50 mPa·s) or greater, and 10000 cP (10000 mPa·s) or less, measured by a rotational viscometer, for example a DVE Digital Viscometer from AMETEK Brookfield in Middleboro, MA, U.S. Rotational viscometers use the idea that the torque required to turn an object in a fluid is a function of the viscosity of that fluid. They measure the torque required to rotate a disk or bob in a fluid at a known speed.

One example of a thermally conductive gel is a gel made from alumina treated with n-decyltrimethoxysilane. Examples of commercially available thermally conductive PCM carriers include DOWSIL™ TC-3015 Reworkable Thermal Gel available from Dow Inc. in Midland, MI, or DOWSIL™ SE4445 thermo-conducting gel, also available from Dow; soft elastomers with liquid metal inclusions (described by Bartlett et al. in High Thermal Conductivity in Soft Elastomers with Elongated Liquid Metal Inclusions, 114 PNAS (2017) 2143-2148); and liquid metal thermal greases such as WT 5932-280 available from Tianjin WaermTimo New Material Technology Corp., Ltd. in Tianjin, China.

In some embodiments, the thermally conductive material in the heat absorbing layer is in the form of particles. The particles may be made of any suitable material that remain flowable at temperatures below the PCM phase change temperature and facilitate absorption of heat by the PCM. That is, the PCM may be in a flowable form. The PCM, when embedded in the thermally conductive material, may be flowable at temperatures above and below the phase change temperature. The PCM may be in flowable particulate form. Examples of suitable materials include salts and minerals. For example, the material may include silicates, such as calcium silicate, aluminum silicate, and the like. Preferably the particles do not absorb water from the surrounding environment. The particles may have any suitable size, such as nanoparticle, microparticle, or a macroparticle. The particles may have a particle size ranging from 10 μm to 5 mm, or from 10 μm to 1 mm.

As discussed, in some embodiments the PCM may be contained in individual modules (also referred to as cells or packets) that can be non-removable (without damaging the cooling article) or selectively inserted and removed from the cooling article. The packets may be filled with particles of PCM embedded in a thermally conductive substrate, such as a thermally conductive PCM carrier. Because the packets may be selectively removed from the cooling article, they may be individually cooled without the need to cool the entire article. For example, the packets may be placed in a cooling device and then inserted back into the cooling article as needed. The packets may include segmented (e.g., via stitching) voids between opposing surfaces of the packet. The packets may be placed along a portion or the entire the length of the article. The article may include one or more openings (e.g., a flap) to facilitate insertion and removal of the packets. The one or more openings (or flaps) may be accessible from the exterior or from the inside of the article (e.g., sleeve). As discussed, in embodiments the openings may be on the interior surface of the cooling article or they can be on exterior surface of the cooling article. The flap openings can be configured to return to a “closed” position after opening or they may be held closed with hook-and-loop (e.g., VELCRO®) straps, clips, snaps, buttons, a zipper, or a combination thereof. The PCM contained in the packets can be inserted into and removed from the segmented void through the flap openings as needed. For example, packets of PCM may be removed and inserted at different intervals to provide for replacement of modules containing PCM having phase change temperature that are the same after changing phases or using PCMs having different phase change temperature at different time intervals as selected by the user. As discussed, the modules or packets can be made from materials that conduct heat, such as fabrics including knitted or woven stainless steel, graphene, carbon nanotube polymers, metals combined with diamond-like coatings (“DLC”) such as DLC coated copper wire, thermally conductive polymers such as COOLPOLY available from Celanese Corporation in Irving, TX, metalized textile fibers (including, for example, silver or copper), such as those available from Statex Produktions- and Vertriebs GmbH in Bremen, Germany (e.g., SHIELDEX® metalized fabrics), and Hengtong Chemical Group Ltd in Jiyuan, China, etc.

In embodiments, the flowable material used to incorporate the PCM material, such as for example the porous silicate particles, can provide a dispersion of local concussive impact momentum and reduce as such the actual impact exerted on, for example, a limb, such as the horse leg. Momentum is defined as Ft (where F is force [Newton] and t is time [second]) and local impact momentum is defined as Ft/A (where A is surface area). Local high impact momentum can be dispersed by the flowable material over a larger surface area, for example over a surface area that is over twice as large or three times as the surface area of the impact. For example, the PCM packets may be provided at some or around the full circumference of a cooling sleeve. This may be particularly beneficial in a cooling sleeve intended for use on a horse's leg. During certain physical activities horse's legs may impact an inanimate object or their hooves or other parts of their legs may impact another part of their leg causing tissue injuries. Gel pads and foams are currently used to provide protection from injury. An advantage of using a herein disclosed flowable material to distribute and absorb local concussive momentum, such as for example silicate porous particles, over soft materials such as gel pads or foams, is that gels and foams may be damaged in absorbing local high momentum, whereas a volume of flowable hard particles is not. The latter as such provides a more durable protection against repeated local concussive impacts. A horse kick momentum can be as high as 400 N·s. This impact momentum can be focused on a surface area as small as one square cm. In embodiments, the PCM packets disclosed can provide protection from concussive injuries by distributing the impact as received by the horse leg over a much larger surface area. In embodiments, the PCM packets may be positioned on the lateral, dorsal and/or palmar/plantar aspect of a cooling sleeve. The PCM packets, including a flowable thermally conductive material (for example, in the shape of hard porous silicate material) therein, may disperse a concussive force directed at a horse's leg. The effect of a concussion momentum damping PCM packet as described can be measured by using two thin film foil force sensors, for example from Sensor Products Inc., 300 Madison Avenue, Madison, NJ 07940 USA. For example, the effect may be measured using the TACTILUS® thin film pressure sensor. One sensor may be located on the outside of the PCM packet and one on the inside of the PCM packet in between the limb (e.g., horse leg) and the PCM. Using a hammer to simulate a horse leg impact, one hits the outer sensor and measures simultaneously the pressure distribution on both inner and outer sensors, giving as such the dispersion properties of the PCM package. According to an embodiment, the area of the cooling article containing the PCM (e.g., the heat absorbing region) is capable of withstanding an impact force of at least 100 N·s, at least 200 N·s, or at least 300 N·s. Although there is no desired upper limit to the amount of impact force the cooling article can withstand, a practical upper limit may be 400 N·s, 600 N·s, or 800 N·s.

The PCM packet may be covered by an outer protective shield or shell (described herein). That is, the PCM packet may be disposed between the protective shield or shell and the leg. The PCM packet may also be used for protection in a cooling article without an outer protective shield.

In some embodiments, the cooling article may include components, materials, or features that reduce or prevent heat from the environment from heating the PCM. This can include insulation, passive cooling material, or both insulation and passive cooling material.

In some embodiments the cooling article may be configured with one or more layers of materials throughout the article or in specific locations within the cooling article. The components that draw heat from the subject may be provided in a layer positioned adjacent the skin of the subject which may be an “inner” layer of the cooling article. The thermally conductive materials may be present in one or more layers, such as an inner layer, an intermediate layer, an outer layer, or any combination thereof. The thermally conductive materials may connect various layers or components of the cooling article, such as the inner layer and the PCM in an intermediate layer. The insulation may be provided in an intermediate layer or an outer layer. The passive cooling material may be provided in an outer layer.

The cooling article may further include features and components that facilitate transfer of heat from the subject to the environment. Specifically, in contrast to regions of the cooling article where close contact between the skin and contact surface of the cooling article is desired, in some embodiments, some regions of a cooling article are spaced away from the skin surface to improve air flow around that region of the body to increase the opportunity for convection cooling. Such features may include spacers on the inner surface of the cooling article to create space between the cooling article and the skin. The spacers may be provided on part of a cooling sleeve. For example, the inner surface of the front/dorsal side of a cooling sleeve that contacts a horse's leg may include spacers between the inner contact surface of the sleeve and the leg to allow sweat or heat radiated from skin and superficial blood vessels to be conveyed to the environment, while the lateral side of the sleeve may include heat absorbing materials, such as a thermally conductive material and/or PCM. In another embodiment, as discussed herein, the underside of a blanket may include spacers to create a space between the contact surface of the blanket and the subject's body to facilitate evaporation of sweat and heat to be conveyed to the environment. Suitable spacers would for example be polymer or wooden beads, for example 9 mm diameter silicon beads available from Silicone Bead Supplies in Harlow, England.

Insulating Materials

The cooling article may further include an insulating layer. The insulating layer may be an intermediate layer between the heat absorbing material and an outer layer. In embodiments, an insulating layer may be an outer layer of the cooling article that insulates the heat absorbing layer from absorbing external heat from the environment. The insulating layer may include any suitable insulating material, such as a polymeric or fibrous material. The insulating material may be a foam. The insulating material may be or may include an elastic material that conforms the cooling article to the body, such as conforming a cooling sleeve to a limb. Examples of suitable insulating materials include THINSULATE®, THERMOLITE®, materials made from an aerogel, polyurethane foam, silicone foam, and the like. The insulating material may be the exterior layer of the cooling article, or the cooling article may include another outer layer, such as a layer of elastic or a protective shield such as a hard shell. In addition, or in alternative embodiments, a cooling article can include a concussion or abrasive resistant outer protective material. In embodiments, the exterior of a cooling article can include a passive cooling material layer.

Aerogels are synthetic porous ultralight materials derived from a gel, in which the liquid component of the gel has been replaced with a gas. The result is a solid with extremely low density and low thermal conductivity. Aerogels can be made from a variety of chemical compounds, such as silica. Examples of suitable aerogels as sheets of various thicknesses include AIRLOY® X110 series materials (e.g., X116 flexible thin films), commercially available from Aerogel Technologies, LLC in Boston, MA. The AIRLOY® X110 series materials are polyimide-based aerogels.

Configuration and Properties of Cooling Article

Passive cooling materials can be used to cool an appendage such as the lower limb of a horse. In an embodiment, the cooling article is a sleeve for cooling a leg of a horse. Horses, in particular performance horses such as race horses, endurance horses or jumpers may experience excessive heating of the lower legs during and/or after an athletic performance. It has been found that the flexor tendons of horses can increase up to 45° C. from “normal” temperature of about 38° C.) after high-speed galloping. The tissues of the leg are highly susceptible to injuries, and the survival rate of cells (e.g., fibroblasts in tendons and skin) in the leg drops sharply at temperatures above 42° C. It is, therefore, desirable to cool the legs or reduce the rate of heating that can occur during exercise. In an embodiment, a tubular sleeve or a sheet of material can be wrapped around the limb and fastened in place using systems for fastening known in the art.

A cooling article provided as a sleeve may be conformed to the contours of the anatomy of a limb or other region of the body, to provide efficient transfer of heat. According to an embodiment, the cooling sleeve is constructed to fit snugly around the desired limb. In embodiments, the cooling sleeve may include a protective shield. In some embodiments the shield may be a protective shell that extends over a significant portion of the perimeter of a cooling sleeve. The term “shield” is used here to refer broadly to protective structures that extend at least partially over a surface of the cooling article. The term “shell” is used here to refer to a protective structure that extends across at least 75% of the surface (e.g., the entire surface) of the cooling article.

According to an embodiment, the cooling article has a layered structure. The cooling article may include a thermally conductive material in a thermally conductive layer and a heat absorbing layer. The cooling article may include an inner layer having a contact surface for placement against the subject, e.g., against the skin of the subject. The cooling article may include an outer layer intended to face the environment when the cooling article is in use. The cooling article may include one or more intermediate layers between the inner layer and the outer layer. In one embodiment, the cooling article includes a heat absorbing layer. The heat absorbing layer may form at least a part of an intermediate layer of the cooling article. The heat absorbing layer may be located throughout the sleeve or only in selected locations. In one embodiment, the cooling article includes one or more thermally conductive materials. The thermally conductive materials may be present in one or more layers, such as the inner layer, the intermediate layer, the outer layer, or any combination thereof. The cooling article may include an insulating layer. The insulating layer may be provided as an intermediate layer. In one embodiment, the cooling article includes one or more passive cooling materials or thermal radiation reflecting materials. The passive cooling or heat reflecting material may form at least a part of the outer layer. In an embodiment, in some regions the passive cooling material may be the only layer. Suitable passive cooling materials are disclosed above.

According to an embodiment, the cooling article is constructed to provide a desired amount of cooling. For example, a cooling sleeve designed to cool a leg (e.g., a calf or thigh) of a human may have a greater cooling capacity than a cooling sleeve designed to cool an arm. On the other hand, a cooling sleeve designed to cool the leg (e.g., suspensory ligament and digital flexor tendons) of a horse may have a greater or lesser cooling capacity than a cooling sleeve designed for human musculature. A cooling blanket may have a cooling capacity designed to lower the core temperature of either a human or an animal. The cooling blanket may be sized to fit the intended subject, such as a human, a large animal (e.g., a horse), or a smaller animal (e.g., a dog).

When the heat absorbing layer of the cooling article is cooled to its phase change temperature or below (e.g., to no more than 1° C. below, no more than 2° C. below, or no more than 5° C. below), the heat absorbing layer may have a cooling capacity of 10 kJ or greater, 15 kJ or greater, 20 kJ or greater, 25 kJ or greater, 30 kJ or greater, 40 kJ or greater, or 50 kJ or greater. The heat absorbing layer may have a cooling capacity of more than 100 kJ, 100 kJ or less, 80 kJ or less, 60 kJ or less, or 40 kJ or less. For example, the heat absorbing layer may have a cooling capacity of 10 kJ to 100 kJ, 10 kJ to 40 kJ, or from 30 kJ to 80 kJ.

The cooling capacity of the heat absorbing layer depends on the PCM(s) selected for the layer, as well as the amount of PCM(s) in the layer. According to an embodiment, the PCM has a cooling capacity of 100 J/g or greater, 150 J/g or greater, 200 J/g or greater, or 250 J/g or greater. The cooling capacity of the PCM does not necessarily have a desired upper end but in practice, the cooling capacity may be up to 500 J/g or up to 400 J/g.

In embodiments, with or without a first layer containing a thermally conductive material, the cooling article may include one or more inflatable elements. The cooling article may include one or more compressible elements or materials. A compressible element or material is one that can absorb a force applied to the element or material by deflecting or deforming. The compressible element may or may not return back to its original shape and size upon removal of the force. Examples of compressible elements include a sand bag, a compression spring, and a piece of foam. The compressibility of a material may be measured as the indentation load deflection or “ILD” which is the amount of pressure or force (in pounds) needed to indent the material by a certain amount (e.g., 25% indentation of a piece of foam having a thickness of 4 inches, using a disc of 50 square inches in size). Suitable compressible materials may have an ILD of 22 to 35. In embodiments that include a hard outer layer or shell (rather than an elastic sleeve), contact with the skin may be improved by inclusion of one or more inflatable or compressible elements. In an embodiment having an inner layer including a thermally conductive material, inflation of the inflatable members or inclusion of a compressible material facilitates increased contact of the contact surface with the skin. In embodiments having a first layer that does not include a thermally conductive material, inflation of the inflatable members or inclusion of a compressible material facilitates increased contact of the heat absorbing layer with the skin. The one or more inflatable elements or compressible material may be disposed between an insulating layer and the heat absorbing layer, or between the hard outer layer or shell and the insulating layer. The inflatable elements can be in the form of one or more inflatable balloons, each having a valve, conduit, and port for inflating and deflating the one or more inflatable balloons. Inflatable elements can be used to press the heat absorbing layer or thermally conductive material against the skin. In embodiments a foam (such as polyurethane foam or silicone foam) can be used as the compressible material to press the heat absorbing layer or thermally conductive material against the skin. The foam can be squeezed while the cooling article (e.g., sleeve) is wrapped around the subject (e.g., a limb, such as a leg). Expansion of the foam will increase contact of the contact surface (either thermally conductive material or heat absorbing layer) against the subject to facilitate heat transfer from the subject. One advantage of using a foam is the additional insulation from external heat as well as being light weight. Various polyurethane foams are commercially available, for example, from Smooth-On, Inc. in Macungie, PA. Various silicone foams are commercially available, for example, from The Rubber Company in Hampshire, United Kingdom. Suitable foams may include open cell foams.

Suitable outer layer materials that may overlay the other layers include materials that have an elastic structure (e.g., a knitted fabric) and/or are made of an elastic material. An example of suitable outer layer material is fabric including elastomeric fibers, such as spandex (e.g., LYCRA®), optionally blended with other fibers, such as synthetic fibers (e.g., polyester, polyamide, polyacrylic, and the like), semi-synthetic fibers (e.g., rayon, modal, lyocell, viscose, and the like), natural fibers (e.g., cotton, hemp, linen, bamboo, wool, and the like), or a combination thereof.

In some embodiments, an insulating layer or outer layer may be further surrounded by materials that are not elastic. For example, at least a portion of the outer layer may include a protective material, such as a protective shield that provides local impact protection. In some embodiments the protective shield is a hard shell that covers a larger portion of the cooling article, such as 20% or more of the perimeter of the cooling article. In some embodiments the protective shell extends over the cooling sleeve in the palmar/plantar region of a horse's limb and in some the shell extends to the lateral and medial surfaces and in some embodiments entirely around the leg. The protective shield preferably comprises a tear and/or puncture-resistant material such as a thermoplastic (e.g., high density or ultra high density polypropylene, polyethylene, polyurethane, or the like), KEVLAR®, GORILLA® glass from Corning Inc. in Corning, NY, flexible ultra-thin glass from Schott AG in Mainz, Germany, flexible ceramic fibers from Eurekite BV in Enschede, The Netherlands, or DYNEEMA®. The protective material may be a breathable material that can also be located around a portion or all of the perimeter of a cooling article. One example of a breathable protective material suitable for the dorsal, lateral or palmar/plantar aspects of a cooling sleeve is a KEVLAR® mesh or a DYNEEMA® Fabric that provides strength and breathability for air flow.

In the case of a cooling sleeve, the insulating layer may extend to the top and/or bottom of the cooling sleeve. For example, the insulating layer may extend to cover the top, bottom, or both top and bottom of the sleeve or sheet of material to insulate the heat absorbing material from the environment when in use. In a tubular sleeve, the insulating layer may form a ring at the top and/or bottom of the sleeve extending above and/or below the heat absorbing layer.

The cooling article may include layers and/or materials that provide the cooling article with beneficial properties in addition to cooling, such as wicking, tear resistance, durability, light weight, breathability, and the like. For example, the cooling article may include a material that provides wicking properties and material that provides strength (e.g., tear resistance and durability). The materials may be configured in separate layers or in the same layer. The cooling article may include a layer or a structure that provides breathability. Exemplary materials that may be used in various layers include natural fibers, synthetic fibers, so-called performance fibers and ultra-strong fibers, metalized fibers, etc. Some fibers may exhibit more than one desired property. For example, a fiber may provide both wicking and strength. Non-limiting exemplary fibers include cotton, abaca, coir, hemp, linen, ramie, jute, kapok, sisal, viscose, rayon, azlon, silk, wool, alpaca, camel hair, carbon fibers, polymeric fibers, such as polyester fibers, polyamide fibers, melamine fibers, polyethylene fibers, polypropylene fibers, polyacrylic fibers, modacrylic fibers, aramid fibers, polybenzimidazole fibers, polyvinyl alcohol fibers, polyvinylidene chloride (SARAN), polyurethane fibers)(SPANDEX®, vinyon fibers, polybenzoxazole (PBO, commercially available as ZYLON® from Toyobo Co., Ltd. in Osaka, Japan), liquid crystal polymer (LCP) polyacrylate (commercially available as VECTRAN® from Kuraray in Tokyo, Japan), and ultra high molecular weight polyethylene (commercially available as SPECTRA® from Honeywell International Inc. in Charlotte, NC, or as DYNEEMA®). The different fibers may be spun together into a yarn and woven, or may be spun separately and woven together. The different fibers may be spun and woven separately and combined (e.g., laminated) as distinct layers. In some embodiments, one or more ultra-strong fibers, such as DYNEEMA®, SPECTRA®, VECTRAN®, or ZYLON®, is used as a reinforcing fiber or layer with another fiber or layer that provides cooling, wicking, breathability, heat transfer, reflectance, or other beneficial property. In one embodiment, one or more ultra-strong fibers, such as DYNEEMA®, SPECTRA®, VECTRAN®, or ZYLON®, is combined with another fiber or layer, such as a plant or animal based fiber (e.g., cotton, abaca, coir, hemp, linen, ramie, jute, kapok, sisal, viscose, rayon, azlon, silk, wool, alpaca, camel hair, or carbon fibers) or a synthetic fiber (polyester fibers, polyamide fibers, melamine fibers, polyethylene fibers, polypropylene fibers, polyacrylic fibers, modacrylic fibers, aramid fibers, polybenzimidazole fibers, polyvinyl alcohol fibers, polyvinylidene chloride (SARAN), polyurethane fibers (SPANDEX®), or vinyon fibers).

The ratio of ultra-strong fibers (DYNEEMA®, SPECTRA®, VECTRAN®, or ZYLON®) to other fiber types in a layer may range from 1 wt-% to 99.9 wt-%. The amount of other types of fibers in the layer may range from 0.01 wt-% to 99 wt-%.

Reinforcing fibers may be distributed unevenly throughout the cooling article. For example, areas prone to tearing, such as edges of the cooling article, may have a higher density of reinforcing fibers.

In some embodiments, in particular those intended for use as horse blankets, the cooling article (e.g., blanket) may include an insect repellant material and/or insect repellant coloring. One or more layers of the cooling article may be impregnated with the insect repellant material, or the insect repellant material may be impregnated or disposed at certain locations on the cooling article. For example, the cooling article may contain pockets to hold reservoirs of repellant materials at locations such as along the topline of the back, or along the edges near the bottom open areas of the cooling article. Examples of suitable insect repellent materials include, for example, naturally occurring pyrethrum extract or a synthetic pyrethroid (such as permethrin, cypermethrin, metofluthrin, profluthrin, or transfluthrin), a naturally occurring oil (such as lemon eucalyptus oil, dove oil, cottonseed oil, neem oil, lemongrass oil, citronella oil, cedarwood lavender oil, rosemary oil, coconut oil, fir needle oil, Litsea cubeba oil, or pyganic (chrysanthemum)oil), terpene (e.g., pulegone), a terpenoid (e.g., geraniol), an alkanoic acid (e.g., a CX-C10-alkanoic acid), an organophosphate (e.g., 2,2-dichlorovinyl dimethyl phosphate), a toluamide (e.g., N,N-diethyl-meta-toluamide (i.e., DEET)), or a combination thereof. Insect repellant coloring or patterns can be used on the outside surface of the article to enhance insect repellant capabilities of the article. Insect repellant coloring may include stripes, for example red and white stripes, on the outside surface of the article.

The cooling article may be provided as a tubular sleeve, as a sheet of material that can be wrapped around the body or a limb, or as a piece of clothing. The sheet of material may be formed including one or more of the layers disclosed herein and include a fastener for holding the sleeve or conforming the sheet in place around the anatomy. Any suitable fastener, including those known in the relevant art, may be used, such as hook-and-loop (e.g., VELCRO®), clips, snaps, buckles, buttons, or a zipper. Examples of suitable fastener systems for equine leg use are known, including from U.S. Pat. No. 6,918,236, the entire disclosure of which is incorporated herein by reference.

In some embodiments, the cooling article is made up of a sheet of material that can be wrapped around the limb. The wrapped sheet may be provided as a single full wrap around the limb, or two or more wraps or three or more wraps around the limb. A single full wrap around the limb refers to the sheet of material wrapping once (100%) around the circumference of the limb. The wrapped sheet may extend 90% to 150% around the limb (about one full wrap), 150% to 250% around the limb (about two full wraps), 250% to 350% around the limb (about three full wraps), or greater. A cooling article intended to be wrapped around a limb more than once may have an inner wrap region that is intended to form the inner layer(s) of wrap, and an outer wrap region that is intended to form the outermost layer of the wrap. The inner wrap region may include thermally conductive material on both sides of the sheet. The inner wrap region may be free of insulating material. The outer wrap region may include thermally conductive material on the inner side only (the side facing the inner wrap region), and insulating material on the outer side (the side facing the outside).

In some embodiments the cooling article is constructed for use with a cast or brace intended for use on a human limb (e.g., arm or leg). The cooling article may be removably attached to the cast or brace. For example, the cooling article may be attached to the cast or brace with an attachment mechanism, such as hook-and-loop (e.g., VELCRO®) straps, clips, snaps, buttons, a zipper, or a combination thereof. The cooling article may be attached when therapeutic cooling is desired, and may be removed when cooling is no longer needed.

In embodiments, the cooling article may be used during training or competition. Weight considerations are important to minimize the pendulum effect of additional weight at the distal end of a limb. The article may be soft and conforming to provide appropriate flexibility and conformability to avoid undesirable pressure points. In embodiments, the cooling article includes a protective shield such as a hard external shell to provide protection from abrasion and/or blunt force impact. For example, a cooling sleeve intended for use during an athletic performance, such as cross-country jumping, may be provided with a protective shield such as a hard protective shell or a regional protective patch. The shell can also encompass the entire circumference of a limb or only selected regions medially, laterally, palmar/plantar of the circumference of a cooling sleeve. The cooling article may be disposed inside the shell and include a heat-conductive layer and a heat absorbing layer or any of the other layers disclosed herein. In embodiments the protective shell may be a regional protective patch and minimally reside only on the palmar/plantar exterior of the boot and extend the entire proximal/distal length of the boot or only is selected regions along the proximal/distal length that is most vulnerable to injury.

In embodiments, the overall weight of the cooling sleeve without a hard shell is 500 g or less, 450 g or less, 400 g or less, 350 g or less, 300 g or less, 250 g or less, 200 g or less, 150 g or less, or 100 g or less. The weight of the cooling sleeve without a hard shell may be 100 g or greater. In some embodiments, the weight of the cooling sleeve without a hard shell is from 100 g to 500 g.

In some embodiments that cooling article may include a temperature sensor coupled with appropriate thin film electronics and a wireless antenna (e.g., BLUETOOTH®) that can continuously measure the temperature within the boot and send information to a user's receiving device (e.g., computer, tablet, smart phone, or the like) to provide real time information on the temperature within the cooling article. The sensor can be applied to either the subject's limb or to the thermally conductive layer, heat absorbing layer, or insulating layer, or each of the foregoing. Examples of suitable sensors include NTC (Negative Temperature Coefficient) thermistors, such as those available from Tayao Technology Co., Ltd. in Taiwan. Another example of a suitable temperature sensor is a BLUETOOTH-enabled temperature measurement patch available as part number TIDA-01524 from Texas Instruments Inc. in Dallas, TX, which includes a high precision digital temperature sensor for sensing skin temperature and a wireless microcontroller (MCU) disposed on a flexible printed circuit board (PCB).

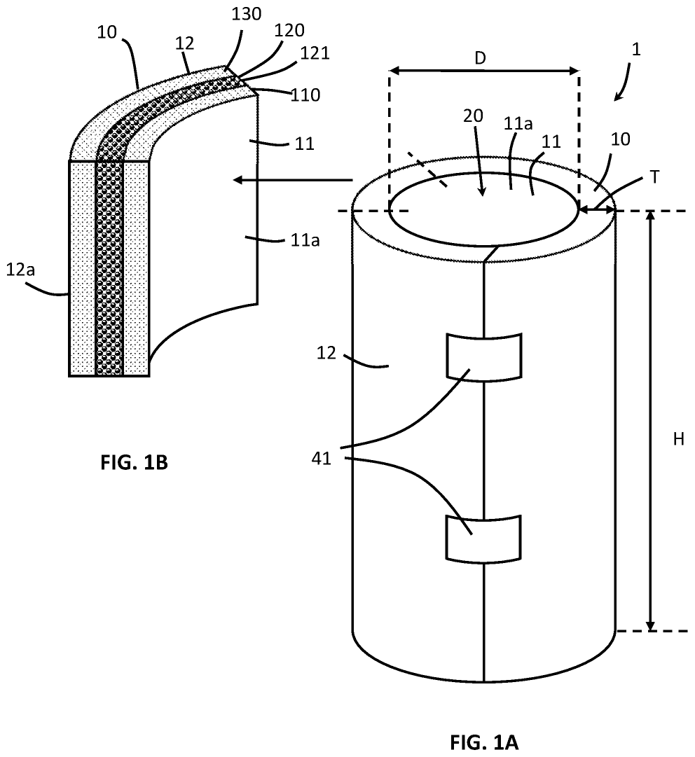

Referring now to FIGS. 1A and 1B, an exemplary embodiment of a cooling article having a tubular form configured as a cooling sleeve 1 is shown. The cooling sleeve 1 has a wall 10 with an inner layer 11 with contact surface 11a and outer layer 12 with outer layer surface 12a. When in a tubular shape the wall 10 defines an interior 20. The wall 10 may be formed as described herein and wrapped into a tubular shape and held in the tubular shape by one or more fasteners 41. The fasteners 41 may include, for example, hook-and-loop (e.g., VELCRO®) straps, clips, snaps, buckles, buttons, a zipper, or a combination thereof. The cooling sleeve 1 may have any suitable inner diameter D to fit snugly to the contours of the anatomy to be cooled. In embodiments, the interior 20 of the cooling sleeve 1 can be configured to conform to the specific contours of the anatomy on which it will be used. The wall 10 may have any suitable thickness T and any suitable height H. The thickness T may be determined by the materials used and by the cooling capacity desired. The inner diameter D and height H may be selected based on the intended use of the cooling sleeve 1. For example, a cooling sleeve 1 for use on a human arm may range from 5 cm to 15 cm in diameter and from 5 cm to 30 cm, or from 8 cm to 20 cm in length. A cooling sleeve 1 for use on a human leg may range from 8 cm to 25 cm in diameter and from 10 cm to 30 cm, or from 15 cm to 25 cm in length. A cooling sleeve 1 for use on a horse leg may range from 5 to 20 cm in diameter and from 15 cm to 45 cm, or from 25 cm to 35 cm in height. In embodiments for use on the lower limb of a horse the sleeve may be of a length to extend the length of the metacarpal (metatarsal) bones from the carpal joint (tarsal joint) to the fetlock or extend distal to the fetlock including the pastern or the pastern and hoof. In embodiments the sleeve may be configured to extend proximal of the carpal (tarsal) joint.

According to an embodiment, the cooling sleeve 1 wall 10 has a layered structure, as shown in FIG. 1B. The wall 10 may include a thermally conductive material in the form of a thermally conductive layer 110, a heat absorbing layer 120, and an insulating layer 130. The thermally conductive layer 110 may be the inner-most layer, forming the inner layer 11 with a contact surface 11a of the sleeve. The insulating layer 130 may be the outer-most layer, forming the outer surface 12 of the sleeve. The heat absorbing layer 120 may be sandwiched between the thermally conductive layer 110 and the insulating layer 130. Although three layers 110, 120, 130 are shown, the wall 10 may include two or more layers or three or more layers. In embodiments, regions of the cooling sleeve may have three layers as described and other regions of the same sleeve have 2, 1 or no materials as will be described below.

According to an embodiment, the heat absorbing layer 120 includes a heat absorbing material, such as a PCM 121.

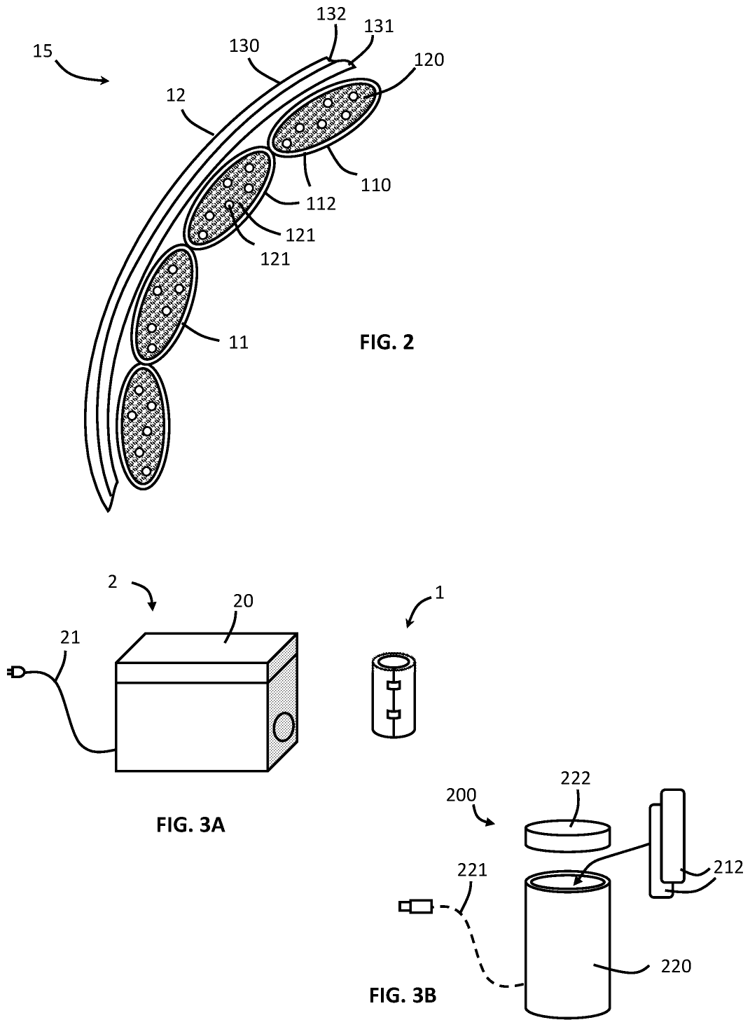

A cross-sectional view of an exemplary wall 15 is shown in FIG. 2. According to an embodiment, thermally conductive layer 110 has a quilted structure forming packets 112 that contain the heat absorbing material. A thermally conductive layer 110 may be made of a material that provides the layer desired properties, such as heat conduction, breathability, and durability, while being capable of maintaining the heat absorbing material in place. In one example, encasement with a thermally conductive layer 110 is made of a metal mesh, such as knitted or woven stainless steel fabric. An exemplary stainless steel fabric may be made from surgical grade stainless steel (e.g., AISI 316L) wire with a 0.004 inch diameter, the fabric having a weight of 190 g/m². Additional examples of the thermally conductive materials include BEKINOX® available from N. V. Bekaert S. A. in Zwevegem, Belgium, thermally conductive polymers such as COOLPOLY® available from Celanese Corporation in Irving, TX, metalized textile fibers (including, for example, silver or copper), such as those available from Statex Produktions- and Vertriebs GmbH in Bremen, Germany (e.g., SHIELDEX® metalized fabrics), and Hengtong Chemical Group Ltd in Jiyuan, China, etc. In embodiments, the thermally conductive layer may not form a packet but rather a continuous matrix material (e.g., open cell foam, closed cell foam, closed cell limestone base, etc.) with the PCM distributed throughout the material. In embodiments the PCM may be encased in a wrap, such as a polymer film, to prevent leaching of the PCM. The thermally conductive layer 110 can be external or internal to the wrap.

The heat absorbing material may include one, two, or more than two PCMs. Examples of PCMs are disclosed throughout this disclosure. In some embodiments the PCM includes PX materials (e.g., PX3, PX5, PX 15) available from Rubitherm Technologies GmbH, in Berlin, Germany. In the embodiment shown, the heat absorbing material includes a first PCM 121 with a first phase change temperature and a second PCM 122 with a second phase change temperature that is different from the first phase change temperature. The PCMs may be mixed with one another and/or include a thermally conductive fluid material such that all materials are dispersed throughout each packet 112.

The insulating layer 130 of a cooling article may include one, two, or more than two layers of materials. In the embodiment shown, the insulating layer 130 includes a first (inner) layer 131 and a second (outer) layer 132. Either the first, second, or both first and second layers may provide insulation. In some embodiments, the first layer 131 primarily provides insulation and the second layer 132 primarily provides the outer surface 12 having desired properties, such as protection of the first layer 131, compression of the cooling sleeve 1 against the limb, a surface for the fastener 41, and/or a desired look and feel. In one example, the first layer 131 is made of an insulating material such as THINSULATE® and the second layer 132 is made of an elastic material such as a spandex-containing fabric.

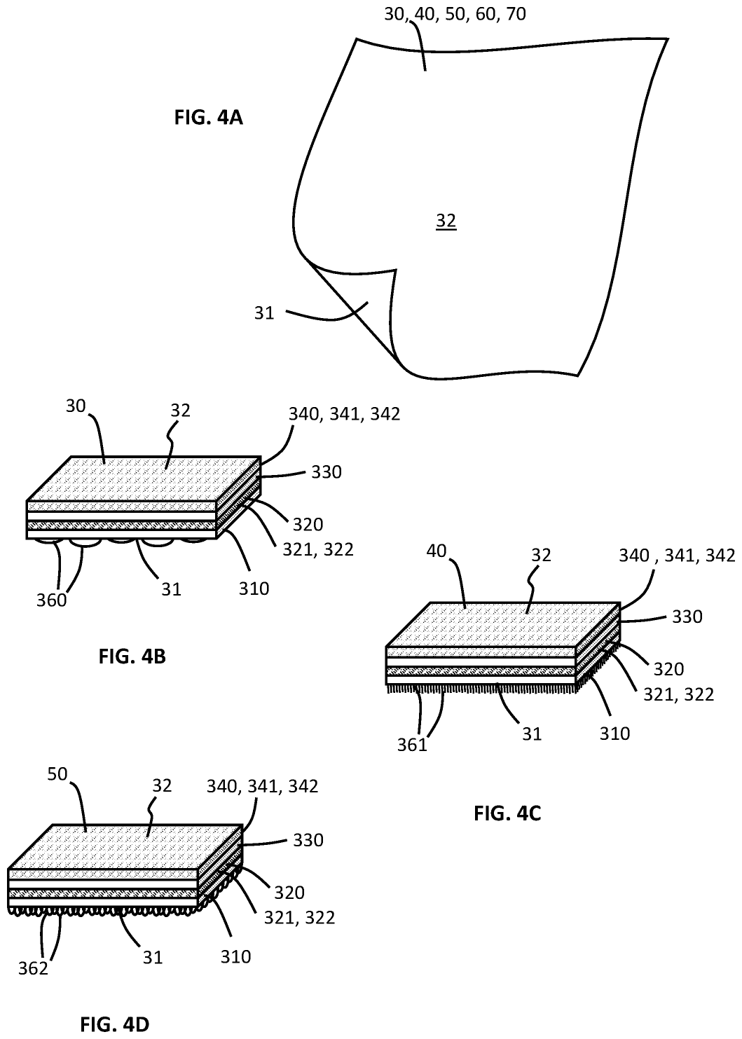

In some cases, it may be desirable to provide the cooling sleeve as part of a portable system that can be used to cool the sleeve and prepare the sleeve for use at home or during travel. The system, schematically shown in FIG. 3A, may include the cooling sleeve 1 as described above, and a portable cooling device 2 (e.g., a cooler). The cooler may include a housing 20 constructed to receive the cooling sleeve and to cool the heat absorbing material (e.g., the PCM) of the cooling sleeve to below the phase change temperature. The portable cooling device 2 may be an electrically powered cooler and may include an electrical connection 21 to a power source. The power source may be a wall outlet, a power outlet in a vehicle, a battery, or the like. The portable cooling device 2 may be sized for cooling one or more cooling sleeves to below the phase change temperature of the PCM. An example of a suitable portable cooling device is the TROPICOOL TC14 available from Dometic Group in Stockholm, Sweden. An alternative example of a portable cooling device 200 is shown in FIG. 3B. The portable cooling device 200 may be shaped as a cylinder 220 with a lid 222. The cylinder 220 may be constructed to accommodate a cooling sleeve 1. Alternatively, the cylinder 220 may be sized and constructed to accommodate a single or more of packets 212 containing the heat absorbing material (e.g., PCM), which may be removed from the cooling sleeve 1 and placed in the portable cooling device 200. A portable cooling device 200 sized to cool only the packets 212 instead of the entire cooling article may have a more convenient small size. The portable cooling device 200 may optionally be cooled by ice, CO₂, nitrogen, or electrically cooled, e.g., using a standard electrical plug or a USB plug 221 or batteries. If more than one PCM is included in the cooling sleeve 1 or the packets 212, the portable cooling device 2, 200 may cool the cooling sleeve to below the lowest phase change temperature. The portable cooling device 2, 200 may include or may be supplemented with additional cooling capacity such that the cooling sleeve 1 or packets 212 may be cooled to any desired temperature, including below 0° C.

The portable cooling device 2, 200 cooled using any suitable cooling system. The portable cooling device 2, 200 may be electrically cooled. The portable cooling device 2, 200 may be chemically cooled using known systems. The portable cooling device 2, 200 may be a passive cooling device. An example of a passive cooling portable cooling device is an insulating container (e.g., a plastic or Styrofoam cooler) that contains a heat sink material that has a capacity to cool the cooling sleeve to the target temperature of the cooling sleeve prior to use. The passive cooling portable cooling device may be shaped like the exemplary cooling devices 2, 200 shown in FIGS. 3A and 3B. Any suitable heat sink material with sufficient cooling capacity may be used, including another PCM with a lower phase change temperature than the cooling sleeve, ice, or gel ice packs, or the like.

A method of cooling the limb of a subject includes cooling the cooling sleeve or PCM packets to a temperature below the phase change temperature of the PCM (e.g., below the lowest phase change temperature if multiple PCMs are used), and applying the cooling sleeve to the limb. The cooling sleeve may be applied by pulling a tubular sleeve onto the limb, wrapping the sleeve around the limb or by wrapping a sheet around the limb and securing the wrapped sheet using the fastener.

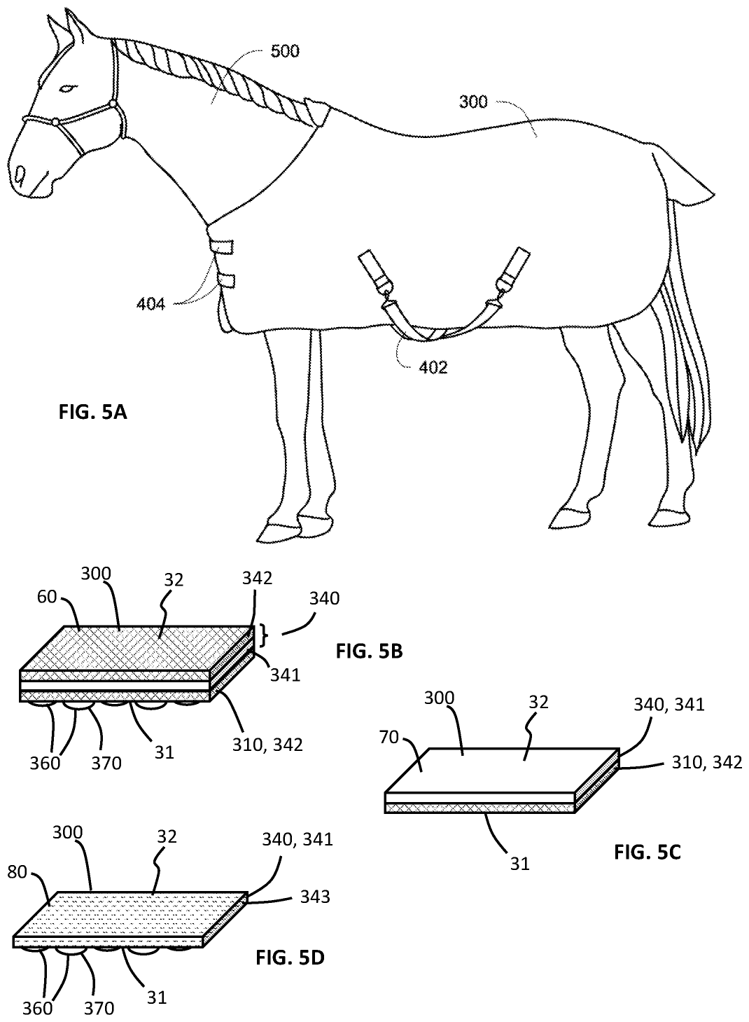

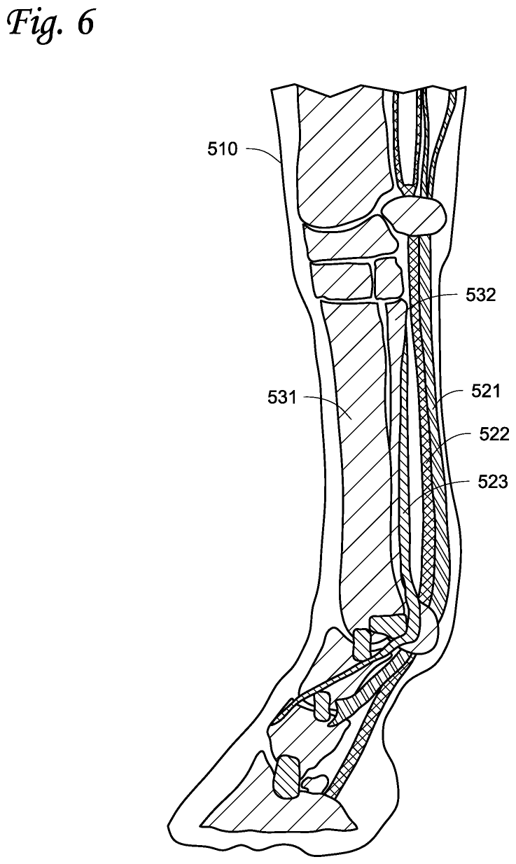

The cooling article 30, 40, 50, 60, 70, and 80 may be provided in the form of a sheet, leg wrap, or a cooling blanket as shown in FIG. 4A or 5A, or a piece of clothing made from a cooling sheet. The cooling article 30, 40, 50, 60, 70, 80 has an inner surface 31, intended to be placed proximate a subject (e.g., human or animal). The cooling article 30, 40, 50, 60, 70, 80 has an outer surface 32 opposite of the inner surface 31. Cross sections of the cooling article 30, 40, 60, 70, 80 according to alternative embodiments, are shown in FIGS. 4B-4D and 5BA-5D.

The cooling article 30, 40, 50 may include multiple layers, including an inner layer 310, a heat absorbing layer 320, an insulating layer 330, and an outer layer 340. The inner layer 310 may include spacers 360 or protrusions 361, 362. In the embodiments shown in FIGS. 4B and 5B, the spacers 360 are ridges extending outward from the inner layer 310. In the embodiment shown in FIG. 4C, the protrusions 361 are fibers extending from the inner layer 310, forming a thermal velvet contact surface. In the embodiment shown in FIG. 4D, the protrusions 362 are loops extending from the inner layer 310, forming a looped pile. The inner layer 310 and the spacers 360 or protrusions 361, 362 may be made from a thermally conductive material. Examples of thermally conductive materials are discussed above. The inner layer 310 and the spacers 360 or protrusions 361, 362 may be constructed from thermally conductive materials to conduct heat from the subject to the heat absorbing layer 320. Alternatively, the spacers 360 may be made of thermally conductive and non-thermally conductive materials, or of non-thermally conductive materials only.

The heat absorbing layer 320 includes PCM 321. The PCM 321 may be provided as particles. The particles of PCM 321 may be embedded in a thermally conductive substrate 322. The thermally conductive substrate 322 may be a gel, a grease, a paste, or a soft elastomer, having a suitable thermal conductivity (e.g., greater than 5 W/(m·K)). The PCM 321 and thermally conductive substrate 322 may further be encased in a thermally conductive material, such as the packets 112 shown in FIG. 2.

The insulating layer 330 may include one, two, or more than two layers of materials. In the embodiment shown, the insulating layer 330 includes a single insulating layer. The insulating layer 330 is made of an insulating material such as THINSULATE® or an aerogel-based material.

The outer layer 340 of the cooling article 30, 40, 50, 60, 70, 80 may form a passive cooling outer layer, as shown. The outer layer 340 may include a passive cooling layer 341 that is made of a passive cooling material. According to an embodiment, the passive cooling material may include a nanoporous polymeric fabric.

In some embodiments, the cooling article 30, 40, 50, 60, 70, 80 includes one or more additional layers or materials that provide additional properties, such as wicking, tear resistance, durability, light weight, breathability, and the like. The cooling article may include a combination of layers and/or materials that contribute to the desired properties. One or more of the layers of the cooling article 30, 40, 50, 60, 70, 80 may include material that provides wicking properties and material that provides strength (e.g., tear resistance and durability). The materials may be provided in separate layers or in the same layer.

In some embodiments, the outer layer 340 further includes a supportive mesh 342 adjacent the passive cooling layer 341. The supportive mesh 342 may form the outer surface 32 of the cooling article 30. Alternatively (or in addition), the supportive mesh 342 may be provided as an inner layer contact surface.

In some embodiments, the passive cooling layer 341 includes reinforcing material, such as reinforcing fibers 343 as part of the passive cooling layer 341, as shown in FIG. 5D.

Exemplary fibers and materials are discussed above and include natural fibers, synthetic fibers, so-called performance fibers and ultra-strong fibers, metalized fibers, etc., and combinations thereof. The different fibers may be spun together into a yarn and woven, or may be spun separately and woven together. The different fibers may be spun and woven separately and combined (e.g., laminated) as distinct layers. In some embodiments, one or more ultra-strong fibers, such as DYNEEMA®, SPECTRA®, VECTRAN®, or ZYLON®, is used as a reinforcing fiber or layer with another fiber (e.g., polyethylene, polypropylene, cotton, etc.) or layer that provides cooling, wicking, breathability, heat transfer, reflectance, or another beneficial property. Reinforcing fibers may be distributed unevenly throughout the cooling article. For example, areas prone to tearing, such as edges of the cooling article 30, 40, 50, 60, 70, 80, may have a higher density of reinforcing fibers.

In FIG. 5A, a schematic view of a cooling article configured as a blanket 300 is shown draped over a horse 500. The cooling blanket 300 may include layers and materials according to the present disclosure. The cooling blanket 300 may include various fasteners, such as bottom straps 402 and front fasteners 404, as shown. The cooling blanket 300 may be a substantially flat sheet that drapes over the body with particular openings and contours that facilitate the head, neck, limbs, etc. In addition to arrangements as shown in sectional views FIGS. 4B-4D, additional sectional views of a cooling blanket 300 are shown in FIGS. 5B-5D. The cooling blanket 300 may include at least an inner layer 310 and an outer layer 340. The inner layer 310 may include spacers 360. The spacers 360 may create space between the skin and the blanket to allow air flow and improved evaporation of sweat. In the embodiment shown in FIG. 5B, the spacers 360 are ridges or nodules 370 extending outward from the inner layer 310. The spacers 360 may be made of any suitable material, such as a polymeric material (e.g., polyurethane, nylon, silicone, etc.) or a naturally occurring material, such as wood. The spacers 360 may include one or more materials. The spacers 360 may be mounted or adhered onto, or integral with the inner layer 310. The outer layer 340 may include a passive cooling layer 341 that is made of a passive cooling material. According to an embodiment, the passive cooling material may include a nanoporous polymeric fabric. In some embodiments, as shown in FIG. 5D, the cooling article 80 may include spacers 360 extending directly from the passive cooling layer 341.

The outer layer 340 may further include a supportive mesh 342 adjacent the passive cooling layer 341. The supportive mesh 342 may form the outer surface 32 of the cooling article 30. Alternatively (or in addition), the supportive mesh 342 may be provided as the inner layer 310, as shown in FIG. 5C. A supportive mesh 342 may be included as part of both the inner layer 310 and the outer layer 340, as shown in FIG. 5B. The supportive mesh 342 may be made, for example, of high density polyethylene (HDPE) or ultra-high molecular weight polyethylene (UHMwPE) or another supportive material as discussed above. In embodiments, reinforcing fibers 343 (e.g., nylon fibers) may be woven into the passive cooling layer 341, as in FIG. 5D. This may eliminate the need for a separate supportive mesh 342. In some embodiments, the cooling article, including layer arrangements shown in FIGS. 4A-4D and 5B-5D, is specifically tailored for use on a horse's leg.

In some embodiments, in particular those intended for use as horse blankets, the cooling article 30, 40, 50, 60, 70, 80 may include an insect repellant material. One or more layers of the cooling article 30, 40, 50, 60, 70, 80 may be impregnated with the insect repellant material, or the insect repellant material may be impregnated or disposed at certain locations on the cooling article 30, 40, 50, 60, 70, 80. For example, the cooling article 30, 40, 50, 60, 70, 80 may contain pockets to hold reservoirs of repellant materials at locations such as along the topline of the back, or along the edges near the bottom open areas of the cooling article 30, 40, 50, 60, 70, 80.

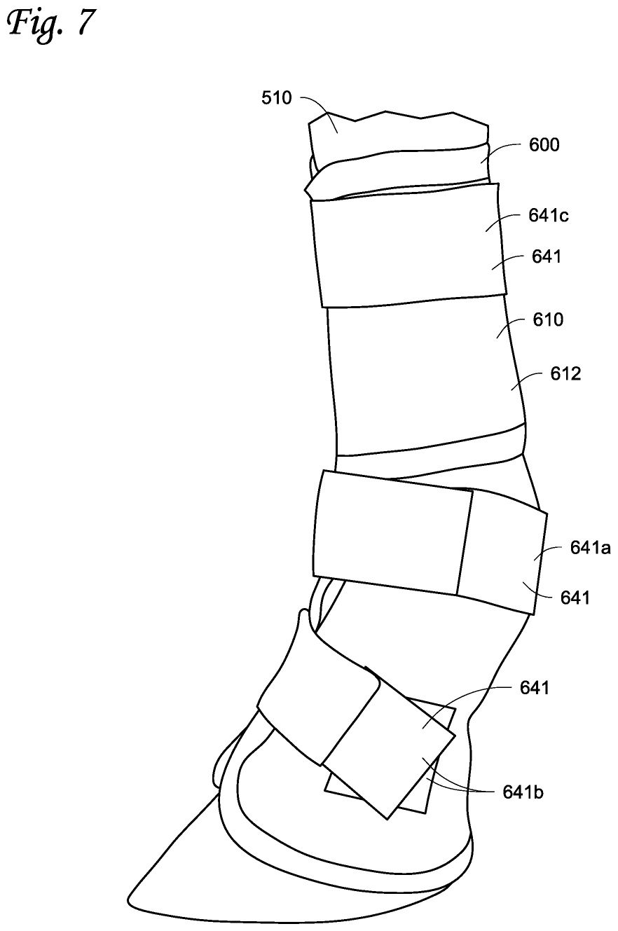

A simplified schematic of equine leg anatomy is shown in FIG. 6. The region of the horse's leg 510 includes various tendons on the back (palmar/plantar) side. The superficial flexor tendon 521, deep digital flexor tendon 522, and suspensory ligament 523 are positioned behind the cannon bone 531 and medial to splint bone 532. These tendons are particularly susceptible to overheating during and after an athletic performance, such as a horse race. While FIG. 6 illustrates a front limb, it will be recognized by one skilled in the art, there are tendon anatomy parallels to the rear limb. In addition, blood supply (not shown) to the tendon structures and the lower limb also flow along the palmar/plantar, lateral, and medial surfaces of the limb. In contrast, the dorsal (front) surface the limb has fewer major vascular structures associated with it. In embodiments, cooling articles configured with different layers and/or materials at different locations for application to a particular appendage can advantageously provide improved cooling efficiencies as well as weight and cost reductions for the article.

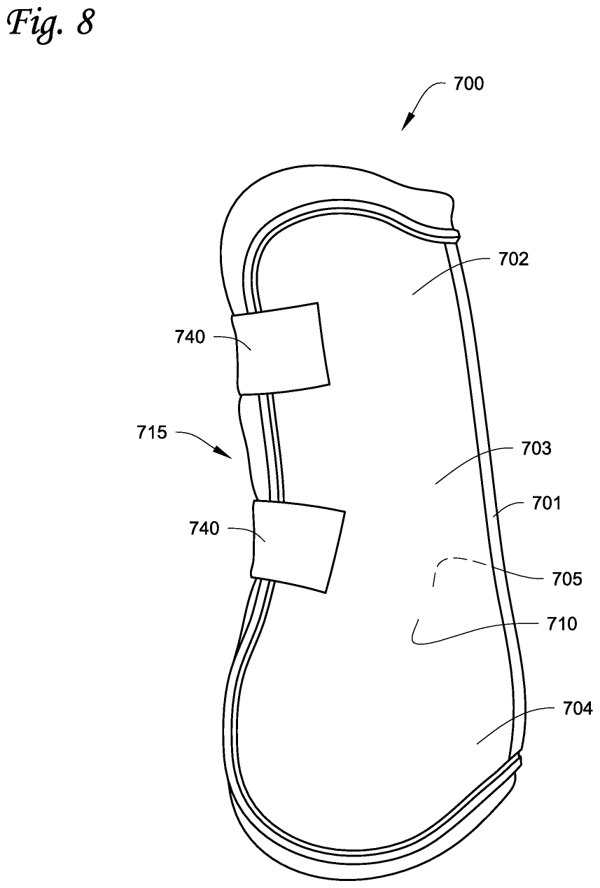

FIG. 7 shows a cooling sleeve 600 configured for a horse's leg 510 according to an embodiment. The cooling sleeve 600 has a wall 610 with an outer surface 612. The wall 610 arranged with one or more layers as has been described herein and formed in a tubular shape conforming to the leg 510 anatomy and secured around the leg 510 by one or more fasteners 641. Any suitable fastener, such as hook-and-loop (e.g., VELCRO®) straps, clips, snaps, buckles, buttons, a zipper, or a combination thereof may be used. In the embodiment shown, three hook-and-loop fasteners 641a-c are positioned in the proximal, middle, and lower regions of the cooling sleeve 600 to provide a secure fit around the leg 510. Such a cooling sleeve arrangement and fasteners is suitable for any of the layer arrangements disclosed here and can be a preferred arrangement for a cooling sleeve using arrangements of layers as disclosed in FIGS. 5B-5D.

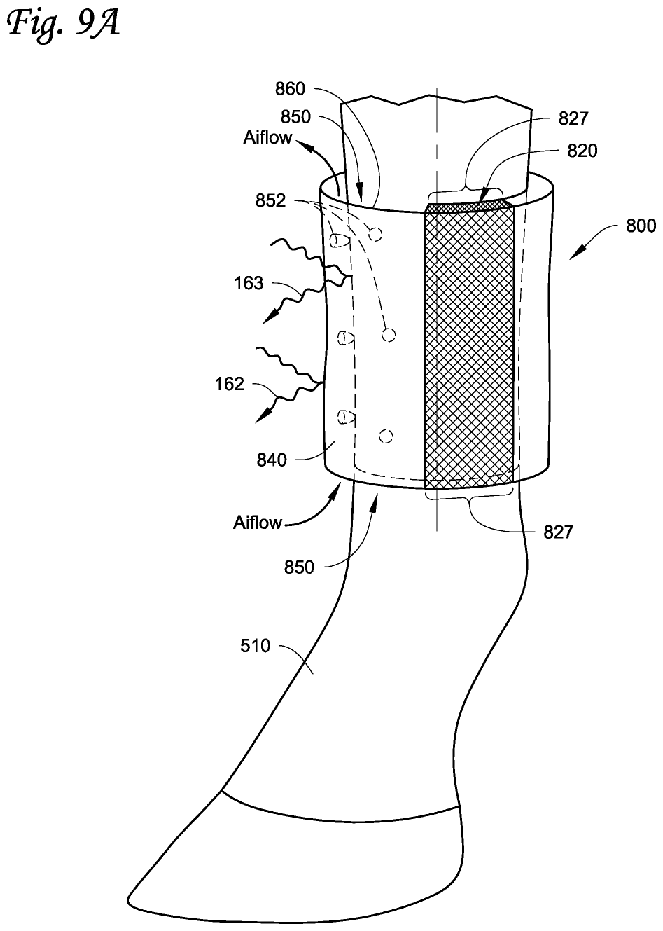

Referring to FIG. 8, in some embodiments, a cooling article, e.g., sleeve 700, can include a hard protective shield 701 positioned as a narrow band on the back (palmar/plantar) aspect of sleeve 700. As discussed above, the protective shield includes a shell that can be located in specific regions along the proximal 702, middle 703 or distal 704 aspect of the sleeve and/or may extend around the medial 705 and/or lateral 710 aspects of the sleeve (not shown) when positioned on a horse's leg 510. In the illustrated embodiment, the front side 715 of the cooling sleeve 700 may be open to facilitate donning and doffing and release of radiant heat from the body. The sleeve 700 may include one or more fasteners 740. Any suitable fastener, such as hook-and-loop (e.g., VELCRO®) straps, clips, snaps, buttons, a zipper, or a combination thereof may be used.

### EXEMPLARY EMBODIMENTS

The following is a listing of exemplary embodiments according to the present disclosure.

According to a first embodiment, a cooling article comprises a thermally conductive layer; and a heat absorbing layer comprising a phase change material having a phase change temperature greater than 0° C.

Embodiment 2 is the cooling article of embodiment 1, wherein the heat absorbing layer has a cooling capacity of 10 kJ or greater, 15 kJ or greater, 20 kJ or greater, 25 kJ or greater, 30 kJ or greater, 40 kJ or greater, or 50 kJ or greater when cooled to no more than 5° C. below the phase change temperature. The heat absorbing layer may have a cooling capacity of 10 kJ to 100 kJ, 10 kJ to 40 kJ, or from 30 kJ to 80 kJ when cooled to no more than 5° C. below the phase change temperature.

Embodiment 3 is the cooling article of embodiment 1 or 2, wherein the heat absorbing layer has a cooling capacity of more than 100 kJ, 100 kJ or less, 80 kJ or less, 60 kJ or less, or 40 kJ or less when cooled to below the phase change temperature.

Embodiment 4 is the cooling article of any one of embodiments 1 to 3, wherein the phase change material has a cooling capacity of 100 J/g or greater, 150 J/g or greater, 200 J/g or greater, or 250 J/g or greater. The cooling capacity may be up to 500 J/g or up to 400 J/g.

Embodiment 5 is the cooling article of any one of embodiments 1 to 4, wherein the heat absorbing layer comprises two or more, three or more, or four or more phase change materials having different phase change temperatures.

Embodiment 6 is the cooling article of embodiment 5, wherein the heat absorbing layer comprises a first phase change material having a phase change temperature in a range of −12° C. to 5° C., from −10 to 5° C., or from −10° C. to 0° C., and a second phase change material having a phase change temperature greater than the first phase change material and in a range of 0° C. to 20° C., from 5° C. to 15° C., from 2° C. to 10° C., or from 4° C. to 8° C.

Embodiment 7 is the cooling article of embodiment 6 further comprising a third phase change material having a phase change temperature greater than the second phase change material and in a range of up to 20° C., or in a range of 4° C. to 30° C., from 6° C. to 20° C., or from 8° C. to 18° C.

Embodiment 8 is the cooling article of any one of embodiments 1 to 7, wherein the heat absorbing layer comprises 50 g or more, 100 g or more, 150 g or more, 200 g or more, or 250 g or more of the phase change material. The heat absorbing layer may include 800 g or less, 600 g or less, 400 g or less, 250 g or less, or 200 g or less of the phase change material.

Embodiment 9 is the cooling article of any one of embodiments 1 to 8, wherein the phase change material is encapsulated.

Embodiment 10 is the cooling article of any one of embodiments 1 to 9, wherein the phase change material is embedded in a thermally conductive material comprising a gel, a grease, a paste, a porous ceramic, a porous mineral, or a soft elastomer, wherein the phase change material is flowable at temperatures above and below the phase change temperature, and optionally wherein the phase change material is in a flowable particulate form.

Embodiment 11 is the cooling article of any one of embodiments 1 to 10, wherein the thermally conductive material exhibits a thermal conductivity of 2 W/(m·K) or greater, 5 W/(m·K) or greater, 10 W/(m·K) or greater, 15 W/(m·K) or greater, or 20 W/(m·K) or greater. The thermal conductivity of the material may be up to 200 W/(m·K) or up to 100 W/(m·K).

Embodiment 12 is the cooling article of any one of embodiments 1 to 11 further comprising an insulating layer. The insulating layer may be an intermediate layer between the heat absorbing material and an outer layer.

Embodiment 13 is the cooling article of embodiment 12, wherein the insulating layer comprises an elastic material.

Embodiment 14 is the cooling article of any one of embodiments 1 to 13, wherein the thermally conductive layer comprises thermally conductive material. The thermally conductive material may comprise metal or material that contains metal, such as stainless steel, aluminum, copper, or an alloy or combination thereof, graphene, carbon nanotube polymer, other thermally conductive polymer.

Embodiment 15 is the cooling article of any one of embodiments 1 to 14, wherein the thermally conductive layer comprises a woven material.

Embodiment 16 is the cooling article of any one of embodiments 1 to 15, wherein the thermally conductive layer forms one or more packets encasing the phase change material.

Embodiment 17 is the cooling article of any one of embodiments 1 to 16 further comprising an inner layer having a contact surface constructed to be placed against a subject to be cooled, the inner layer comprising a thermally conductive material. The thermally conductive material may comprise metal or material that contains metal, such as stainless steel, aluminum, copper, silver, or an alloy or combination thereof, graphene, boron nitride, carbon nanotube polymer, other thermally conductive polymer.

Embodiment 18 is the cooling article of embodiment 17, wherein the contact surface comprises a plurality of protrusions extending from an inner surface of the cooling article, the plurality of protrusions comprising a thermally conductive material.

Embodiment 19 is the cooling article of embodiment 18, wherein the plurality of protrusions comprise ridges, loops, or fibers forming a thermal velvet.

Embodiment 20 is the cooling article of any one of embodiments 17 to 19, wherein the thermally conductive material exhibits a thermal conductivity of 10 W/(m·K) or greater, 15 W/(m·K) or greater, or 20 W/(m·K) or greater. The thermal conductivity of the material may be up to 200 W/(m·K) or up to 100 W/(m·K).

Embodiment 21 is the cooling article of any one of embodiments 1 to 20, wherein the cooling article comprises a sheet of material formed by the layers, and a fastener constructed to hold the sheet of material in a tubular shape.

Embodiment 22 is the cooling article of any one of embodiments 1 to 21, wherein the cooling article is a sleeve conformable around a limb.

Embodiment 23 is the cooling article of embodiment 22, wherein the sleeve is encased in a protective outer shell.

Embodiment 24 is a system for providing cooling to a limb, the system comprising a cooling article according to any one of embodiments 1 to 23; and a portable cooling device comprising a housing constructed to receive the cooling article and to cool the phase change material of the cooling article to below the phase change temperature.

Embodiment 25 is the system of embodiment 24, wherein the portable cooling device comprises an electrically powered cooler and an electrical connection to a power source.

Embodiment 26 is a heat absorbing composition comprising a thermally conductive substrate; and a phase change material dispersed in the thermally conductive substrate.

Embodiment 27 is the heat absorbing composition of embodiment 26, wherein the thermally conductive substrate has a thermal conductivity of 2 W/(m·K) or greater, 5 W/(m·K) or greater, 10 W/(m·K) or greater, 15 W/(m·K) or greater, or 20 W/(m·K) or greater. The thermal conductivity of the material may be up to 200 W/(m·K) or up to 100 W/(m·K).

Embodiment 28 is the heat absorbing composition of embodiment 26, wherein the thermally conductive substrate comprises a gel, a grease, a paste, a porous ceramic, a porous mineral, or a soft elastomer, wherein the phase change material is flowable at temperatures above and below the phase change temperature, and optionally wherein the phase change material is in a flowable particulate form. The thermally conductive substrate may comprise a gel comprising alumina treated with n-decyltrimethoxysilane.

Embodiment 29 is a cooling article comprising a sleeve, the sleeve comprising the heat absorbing composition of embodiment 27 or 28 provided in a heat absorbing layer.

Embodiment 30 is the cooling article of embodiment 29, wherein the phase change material has a phase change temperature, and wherein the heat absorbing layer has a cooling capacity of 10 kJ or greater, 15 kJ or greater, 20 kJ or greater, 25 kJ or greater, 30 kJ or greater, kJ or greater, or 50 kJ or greater when cooled to no more than 5° C. below the phase change temperature. The heat absorbing layer may have a cooling capacity of 10 kJ to 100 kJ, 10 kJ to kJ, or from 30 kJ to 80 kJ when cooled to no more than 5° C. below the phase change temperature.

Embodiment 31 is the cooling article of embodiment 30, wherein the heat absorbing layer has a cooling capacity of more than 100 kJ, 100 kJ or less, 80 kJ or less, 60 kJ or less, or 40 kJ or less when cooled to below the phase change temperature.

Embodiment 32 is the cooling article of any one of embodiments 29 to 31, wherein the phase change material has a cooling capacity of 100 J/g or greater, 150 J/g or greater, 200 J/g or greater, or 250 J/g or greater. The cooling capacity may be up to 500 J/g or up to 400 J/g.

Embodiment 33 is the cooling article of any one of embodiments 29 to 32, wherein the heat absorbing layer comprises two or more, three or more, or four or more phase change materials having different phase change temperatures.

Embodiment 34 is the cooling article of embodiment 33, wherein the heat absorbing layer comprises a first phase change material having a phase change temperature in a range of −12° C. to 5° C., from −10 to 5° C., or from −10° C. to 0° C., and a second phase change material having a phase change temperature greater than the first phase change material and in a range of 0° C. to 20° C., from 5° C. to 15° C., from 2° C. to 10° C., or from 4° C. to 8° C.

Embodiment 35 is the cooling article of embodiment 34 further comprising a third phase change material having a phase change temperature greater than the second phase change material and in a range of up to 20° C., or in a range of 4° C. to 30° C., from 6° C. to 20° C., or from 8° C. to 18° C.

Embodiment 36 is the cooling article of any one of embodiments 29 to 35, wherein the heat absorbing layer comprises 50 g or more, 100 g or more, 150 g or more, 200 g or more, or 250 g or more of the phase change material. The heat absorbing layer may include 800 g or less, 600 g or less, 400 g or less, 250 g or less, or 200 g or less of the phase change material.

Embodiment 37 is the cooling article of any one of embodiments 29 to 36, wherein the phase change material is encapsulated.

Embodiment 38 is the cooling article of any one of embodiments 29 to 37, wherein the phase change material is embedded in a thermally conductive material comprising a gel, a grease, a paste, a porous ceramic, a porous mineral, or a soft elastomer, wherein the phase change material is flowable at temperatures above and below the phase change temperature, and optionally wherein the phase change material is in a flowable particulate form.

Embodiment 39 is the cooling article of any one of embodiments 29 to 38 further comprising an insulating layer. The insulating layer may be an intermediate layer between the heat absorbing material and an outer layer.

Embodiment 40 is the cooling article of embodiment 39, wherein the insulating layer comprises an elastic material.

Embodiment 41 is the cooling article of any one of embodiments 29 to 40 further comprising a thermally conductive layer comprising stainless steel.

Embodiment 42 is the cooling article of embodiment 41, wherein the thermally conductive layer comprises a woven material.

Embodiment 43 is the cooling article of any one of embodiments 41 to 42, wherein the thermally conductive layer forms one or more packets encasing the phase change material.

Embodiment 44 is the cooling article of any one of embodiments 29 to 44, further comprising an inner layer constructed to be placed against a subject to be cooled, the inner layer comprising a thermally conductive material. The thermally conductive material may comprise metal or material that contains metal, such as stainless steel, aluminum, copper, or an alloy or combination thereof, graphene, carbon nanotube polymer, other thermally conductive polymer.

Embodiment 45 is the cooling article of embodiment 44, wherein the inner layer comprises a plurality of protrusions extending from an inner surface of the cooling article, the plurality of protrusions comprising a thermally conductive material.

Embodiment 46 is the cooling article of embodiment 45, wherein the plurality of protrusions comprise ridges, loops, or fibers forming a thermal velvet.

Embodiment 47 is the cooling article of any one of embodiments 39 to 46, wherein the cooling article comprises a sheet of material formed by the layers,

Embodiment 48 is the cooling article of any one of embodiments 29 to 47, wherein the article comprises a fastener constructed to hold the sheet of material in a tubular shape.

Embodiment 49 is the cooling article of any one of embodiments 29 to 48, wherein the sleeve is encased in a protective outer shell.

Embodiment 50 is a cooling article comprising a passive cooling layer having a first surface and a second surface; and a supportive mesh adjacent the first surface, wherein the cooling article is constructed to conform to a portion of a subject's body.

Embodiment 51 is the cooling article of embodiment 50, wherein the cooling article is constructed to conform to a torso of the subject's body.

Embodiment 52 is the cooling article of embodiment 50, wherein the cooling article is constructed to conform to an appendage of the subject's body.

Embodiment 53 is the cooling article of embodiment 52, wherein the appendage is a leg.

Embodiment 54 is the cooling article of embodiment 50 further comprising a second supportive mesh adjacent the second surface.

Embodiment 55 is the cooling article of embodiment 50 further comprising one or more fasteners constructed to maintain the cooling article in position on the subject's body.

Embodiment 56 is a cooling article comprising a sleeve constructed to be applied on a limb. The sleeve comprises a dorsal region positionable adjacent a dorsal aspect of the limb; a palmar/plantar region positionable adjacent a palmar aspect of the limb; a medial region positionable adjacent a medial aspect of the limb; a lateral region positionable adjacent a lateral aspect of the limb; a heat absorbing region that is present in one or more of the palmar/plantar, medial, and lateral regions; a heat convective region that is present in the dorsal region; an inner layer forming a contact surface with the limb; an outer layer; and a thermally conductive layer disposed in the heat absorbing region between the inner layer and the outer layer. The thermally conductive layer comprises a carrier comprising a thermally conductive substrate; and a phase change material dispersed in the thermally conductive substrate. The outer layer comprising an insulating layer adjacent the thermally conductive layer, and a passive cooling layer.

Embodiment 57 is the cooling article of embodiment 56, wherein the insulating layer comprises an elastic layer.

Embodiment 58 is the cooling article of embodiment 56 or 57, wherein the passive cooling layer is disposed on a portion of an outer surface of the cooling article.

Embodiment 59 is the cooling article of any one of embodiments 56 to 58, wherein the passive cooling layer surrounds the cooling article.

Embodiment 60 is the cooling article of any one of embodiments 56 to 59, wherein the passive cooling layer is disposed in the dorsal region.

Embodiment 61 is the cooling article of any one of embodiments 56 to 60, wherein the passive cooling layer is the outermost layer.

Embodiment 62 is the cooling article of any one of embodiments 56 to 61, wherein the outer layer further comprises a protective shell in the palmar/plantar region.

Embodiment 63 is the cooling article of embodiment 62, wherein the protective shell extends along the entire length of the cooling article.

Embodiment 64 is the cooling article of any one of embodiments 56 to 63, further comprising one or more fasteners to fasten the sleeve on or around the limb.

Embodiment 65 is the cooling article of any one of embodiments 56 to 64, wherein the contact surface comprises a plurality of protrusions in the heat convective region.

Embodiment 66 is a cooling article comprising a passive cooling layer and reinforcing fibers configured to support the passive cooling layer, wherein the cooling article is configured to conform to a portion of a subject's body. The reinforcing fibers may be woven into the passive cooling layer. The reinforcing fibers may be disposed adjacent the passive cooling layer.

Embodiment 67 is the cooling article of embodiment 66 further comprising a heat absorbing composition arranged as a heat absorbing layer, the heat absorbing composition comprising a thermally conductive substrate; and a phase change material dispersed in the thermally conductive substrate.

Embodiment 68 is the cooling article of embodiment 67, wherein the thermally conductive substrate has a thermal conductivity of 2 W/(m·K) or greater, 5 W/(m·K) or greater, W/(m·K) or greater, 15 W/(m·K) or greater, or 20 W/(m·K) or greater. The thermal conductivity of the material may be up to 200 W/(m·K) or up to 100 W/(m·K).

Embodiment 69 is the cooling article of embodiment 67 or 68, wherein the thermally conductive substrate comprises a gel, a grease, a paste, a porous ceramic, a porous mineral, or a soft elastomer, wherein the phase change material is flowable at temperatures above and below the phase change temperature, and optionally wherein the phase change material is in a flowable particulate form, optionally wherein the phase change material comprises a gel comprising alumina treated with n-decyltrimethoxysilane.

Embodiment 70 is the cooling article of any one of embodiments 67 to 29, wherein the cooling article is formed as a sleeve or a blanket.

Embodiment 71 is the cooling article of any one of embodiments 67 to 70, wherein the phase change material has a phase change temperature, and wherein the heat absorbing layer has a cooling capacity of 10 kJ or greater, 15 kJ or greater, 20 kJ or greater, 25 kJ or greater, 30 kJ or greater, 40 kJ or greater, or 50 kJ or greater when cooled to no more than 5° C. below the phase change temperature. The heat absorbing layer may have a cooling capacity of 10 kJ to 100 kJ, 10 kJ to 40 kJ, or from 30 kJ to 80 kJ when cooled to no more than 5° C. below the phase change temperature.

Embodiment 72 is the cooling article of any one of embodiments 67 to 71, wherein the heat absorbing layer has a cooling capacity of more than 100 kJ, 100 kJ or less, 80 kJ or less, 60 kJ or less, or 40 kJ or less when cooled to below the phase change temperature.

Embodiment 73 is the cooling article of any one of embodiments 67 to 72, wherein the phase change material has a cooling capacity of 100 J/g or greater, 150 J/g or greater, 200 J/g or greater, or 250 J/g or greater. The cooling capacity may be up to 500 J/g or up to 400 J/g.

Embodiment 74 is the cooling article of any one of embodiments 67 to 73, wherein the heat absorbing layer comprises two or more, three or more, or four or more phase change materials having different phase change temperatures.

Embodiment 75 is the cooling article of any one of embodiments 67 to 74, wherein the heat absorbing layer comprises a first phase change material having a phase change temperature in a range of −12° C. to 5° C., from −10 to 5° C., or from −10° C. to 0° C., and a second phase change material having a phase change temperature greater than the first phase change material and in a range of 0° C. to 20° C., from 5° C. to 15° C., from 2° C. to 10° C., or from 4° C. to 8° C.

Embodiment 76 is the cooling article of any one of embodiments 72 to 75 further comprising a third phase change material having a phase change temperature greater than the second phase change material and in a range of up to 20° C., or in a range of 4° C. to 30° C., from 6° C. to 20° C., or from 8° C. to 18° C.

Embodiment 77 is the cooling article of any one of embodiments 67 to 76, wherein the heat absorbing layer comprises 50 g or more, 100 g or more, 150 g or more, 200 g or more, or 250 g or more of the phase change material. The heat absorbing layer may include 800 g or less, 600 g or less, 400 g or less, 250 g or less, or 200 g or less of the phase change material.

Embodiment 78 is the cooling article of any one of embodiments 67 to 77, wherein the phase change material is encapsulated.

Embodiment 79 is the cooling article of any one of embodiments 67 to 78, wherein the phase change material is embedded in a thermally conductive material comprising a gel, a grease, a paste, or a soft elastomer.

Embodiment 80 is the cooling article of any one of embodiments 66 to 79 further comprising an insulating layer. The insulating layer may be an intermediate layer between the heat absorbing material and an outer layer.

Embodiment 81 is the cooling article of embodiment 80, wherein the insulating layer comprises an elastic material.

Embodiment 82 is the cooling article of any one of embodiments 67 to 81, wherein the thermally conductive layer comprises stainless steel.

Embodiment 83 is the cooling article of any one of embodiments 67 to 82, wherein the thermally conductive layer comprises a woven material.

Embodiment 84 is the cooling article of any one of embodiments 67 to 83, wherein the thermally conductive layer forms one or more packets encasing the phase change material.

Embodiment 85 is the cooling article of any one of embodiments 66 to 84, further comprising an inner layer constructed to be placed against a subject to be cooled, the inner layer comprising a thermally conductive material. The thermally conductive material may comprise metal or material that contains metal, such as stainless steel, aluminum, copper, or an alloy or combination thereof, graphene, carbon nanotube polymer, other thermally conductive polymer.

Embodiment 86 is the cooling article of embodiment 85, wherein the inner layer comprises a plurality of protrusions extending from an inner surface of the cooling article, the plurality of protrusions comprising a thermally conductive material.

Embodiment 87 is the cooling article of embodiment 86, wherein the plurality of protrusions comprise ridges, loops, or fibers forming a thermal velvet.

Embodiment 88 is the cooling article of any one of embodiments 67 to 87, wherein the cooling article comprises a fastener constructed to hold the cooling article in a tubular shape.

Embodiment 89 is the cooling article of any one of embodiments 67 to 88, wherein the cooling article is a sleeve encased in a protective outer shell.

Embodiment 90 is an equine leg boot comprising a dorsal region positionable adjacent a dorsal aspect of the limb; a palmar/plantar region positionable adjacent a palmar/plantar aspect of the limb; a medial region positionable adjacent a medial aspect of the limb; a lateral region positionable adjacent a lateral aspect of the limb; a heat absorbing region that is present in one or more of the palmar/plantar, medial, and lateral regions; the heat absorbing region comprising a phase change material having a phase change temperature, wherein the heat absorbing region is compliant at temperatures above and below the phase change temperature.

Embodiment 91 is the equine leg boot of embodiment 90, including any of the features of the preceding embodiments.

Embodiment 92 is the equine leg boot of embodiment 90 or 91, wherein the phase change material is disposed within a packet, where in the packet may be selectively removed from the equine leg boot.

Embodiment 93 is the equine leg boot of any one of embodiments 90 to 92, wherein the packets comprise a thermally conductive layer.

Embodiment 94 is the equine leg boot of any one of embodiments 90 to 93, wherein the phase change material is dispersed within a carrier, optionally wherein the carrier comprises a gel, a grease, a paste, a porous ceramic, a porous mineral, or a soft elastomer.

Embodiment 95 is the equine leg boot of any one of embodiments 90 to 94 further comprising a protective shield.

Embodiment 96 is the equine leg boot of any one of embodiments 90 to 95 further comprising an insulating layer.

### Example 1

A cooling sleeve for use on a horse's leg may be constructed as follows.

Estimating the desired cooling capacity: The heat capacity of a muscle is estimated at 3500 J/kg/° C. The density of muscle is about 1.06 kg/L. The desired temperature change from ° C. to 38° C. is 7° C. The volume of the leg is estimated based on a cylinder of 30 cm height and a diameter of 8 cm, calculating to a volume of 1507 cm³. It can be estimated that the bone takes ⅓ of that space. Thus, the section of leg being cooled is estimated to include approximately 1 kg of muscle. Based on these estimates, the amount of heat energy to be removed from the muscle is

$$7 ° C . \cdot 1 kg \cdot {3500}^{\frac{J}{kg}} / ° C . = 24500 J = 24.5 kJ .$$

The PCM selected for the sleeve is CRODATHERM 21 having a phase change temperature of 21° C. CRODATHERM 21 has a heat capacity at phase change of 190 kJ/kg. The amount needed for absorbing 24.5 kJ is 130 g.

The density of CRODATHERM 21 is 850 g/dm³. Thus, the calculated volume of CRODATHERM 21 is 0.15 dm³. Spread across the 30 cm height of the cylinder, the volume of CRODATHERM 21 is 5 cm³ per 1 cm cylinder height. The thickness of CRODATHERM 21 will be about 2 mm.

To the approximately 2 mm layer of PCM, a 2 mm thickness insulating outer layer may be added. The resulting sleeve may be 4-5 mm thick, and suitable for short term or long term use.

### Example 2

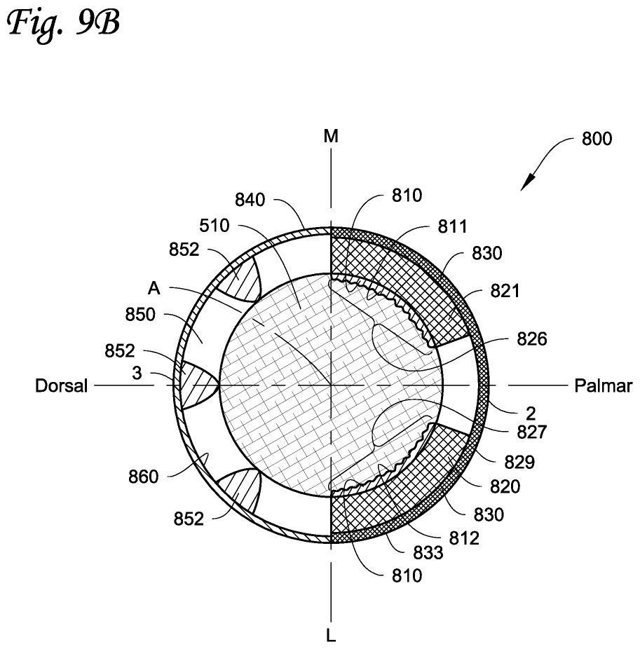

Referring to FIGS. 9A and 9B, a cooling article in the form of a sleeve 800 for a lower equine limb 510 is shown. In the illustrated embodiment the heat absorbing layer 820, 821 is located within the article positioned at the lateral (L) and medial (M) aspects of the tendons, respectively. The thermally conductive layer 810 as described above creates an inner medial layer 811 and an inner lateral layer 812 providing a contact surface in those regions of the cooling article. The heat absorbing layer 820 may include a PCM 821 with a thermally conductive flowable carrier (not shown). On the medial (M), lateral (L), and palmar aspects, the heat absorbing regions 826 and 827 are surrounded by an outer layer 829 including an insulating layer 830. The insulating layer 830 may optionally include an elastic layer 833 or have elastic material included in the insulating layer. The insulating layer 830 may also include a layer of foam, such as polyurethane foam or silicone foam. The foam may be an additional layer between the elastic layer 833 and the heat absorbing layer 820. In some embodiments, a passive cooling layer 840 can be included that provides passive cooling by reflecting radiation 162 of the sun and releasing infra-red heat 163 from the limb. The passive cooling layer 840 may be disposed on a portion (as shown in FIGS. 9A-9B) of the cooling article or may surround the entire cooling article. The passive cooling layer 840, where present, may be the outermost layer. For further protection, some or a portion of the palmar (back) surface of the cooling article may include a protective shield such as a hard shell (not shown in FIGS. 9A-B) to protect the tendons from injury (see, e.g., the hard protective shield 701 in FIG. 8). The hard shell may extend partially along the length (height) of the cooling article or may extend the entire length of the cooling article. In embodiments, the passive cooling layer 840 may only be located in the dorsal region of the cooling article (as shown in FIG. 9A-9B) or around some or all of the circumference of the cooling article.

The cooling article may include a plurality of spacers 852 protruding from the inner surface of the cooling article. The spacers 852 may be constructed to reduce direct contact of the passive cooling layer 840 and the dorsal surface of the limb 510 to facilitate convective cooling. The spacers 852 create a gap 850 to be formed between the inner surface 860 of the passive cooling layer 840 and the surface outer surface of the horse's leg 510. Airflow through the gap 850 can facilitate cooling of the leg 510. The spacers 852 may be protrusions protruding from the inner surface 860 of the cooling article. As illustrated in FIGS. 9A-9B, in some embodiments, one or both of the thermally conductive layer 810 and the heat absorbing layer 820 can be excluded at the dorsal (front) side of the cooling article. The cooling sleeve 800 may include fasteners to fasten the sleeve on or around the limb 510 (see, e.g., fasteners 740 shown in FIG. 8). The fasteners may include hook-and-loop (e.g., VELCRO®), clips, snaps, buttons, a zipper, or a combination thereof. In some embodiments only fastening structures are present on the dorsal aspect of the limb further permitting direct airflow on the dorsal surface of the limb.

### Example 3

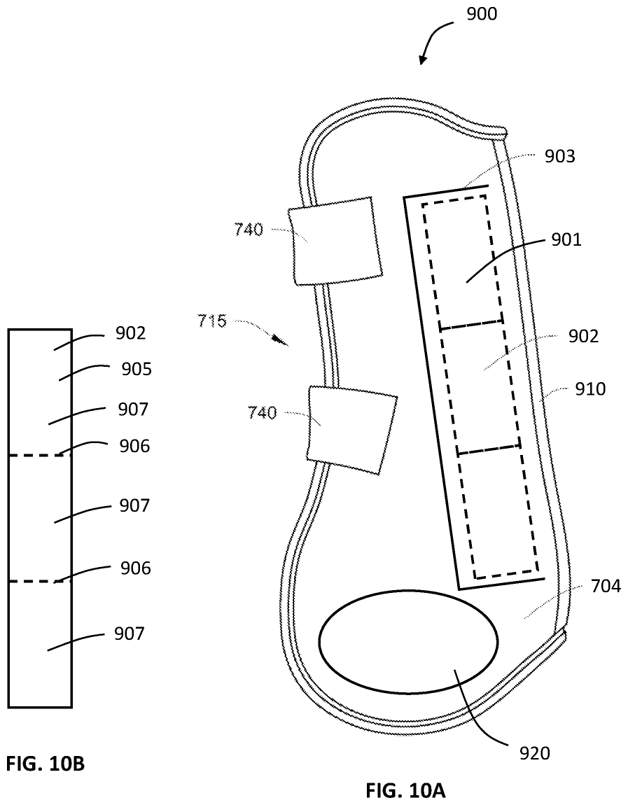

Referring to FIGS. 10A and 10B, a cooling article in the form of an equine leg boot 900 for a lower equine limb 510 is shown. In the illustrated embodiment the heat absorbing layer 901, shown in phantom lines, is in the form of a PCM packet 902 beneath flap 903 that can be opened and closed to insert or remove PCM packet 902. In this embodiment a second heat absorbing layer is present but not shown on the opposite side of equine leg boot 900. PCM packet 902 includes a thermally conductive material 905 that encloses a flowable PCM such as PX 3, PX 5, PX15 or other flowable PCM as is available from Rubitherm Technologies GmbH, Berlin, Germany. In the illustrated embodiment, the thermally conductive material includes stitching 906 that divides PCM packet 902 into three separate segments 907. Any suitable number of segments 907 could be used as desired to minimize shifting of the PCM within the packet and maintain conformability for comfort and proximate fit to the relevant anatomy. While not required, such segmenting reduces the chances of the flowable PCM shifting due to gravity. Behind flap 903 there is a compartment (not shown) within which PCM packet 902 is located during use. The inside wall of the compartment (not shown) is also formed of a thermally conductive material that forms the contact surface which contacts the horse's leg. Flap 903 can be secured to the outside of the boot using hook and loop fastening, straps, belts, buckles, or other known systems for securing a flap. Alternatively, the configuration of flap 903 can be such that the flap configuration provides for the flap to remain in a closed position unless displaced for inserting or removing PCM packet 902. Flap 903 can be a single layer or multiple layers comprising a thermally conductive layer, insulating layer and/or elastic layer as described previously for other embodiments. In embodiments not illustrated, rather than a flap that opens to the outside of the boot, the flap or other selective access port can be located on the inner surface of the boot so the PCM packet 902 is inserted and removed from the inside. An advantage of the location of the flap on the illustrated embodiment is that it provides for access to the PCM packet 902 without removing the boot from the horse's leg.

For further protection, some or a portion of the palmar (back) surface of the cooling article may include a protective shield 910, such as a KEVLAR® strip to protect the palmar/plantar aspect of the tendons. In addition, a lateral shield 920, also made from a material that protects from blunt trauma, is located on the distal portion of equine leg boot 900. A similar protective shield can be located on the medial side of the boot. Suitable materials include tear and/or puncture-resistant materials such as a thermoplastic (e.g., high density or ultra high density polypropylene, polyethylene, polyurethane, or the like), KEVLAR®, GORILLA® glass, flexible ultra-thin glass, flexible ceramic fibers, and DYNEEMA®. The protective material may be a breathable material that can also be located around a portion or all of the perimeter of a cooling article.

All references and publications cited herein are expressly incorporated herein by reference in their entirety into this disclosure, except to the extent they may directly contradict this disclosure. Although specific embodiments have been illustrated and described herein, it will be appreciated by those of ordinary skill in the art that a variety of alternate and/or equivalent implementations can be substituted for the specific embodiments shown and described without departing from the scope of the present disclosure. It should be understood that this disclosure is not intended to be unduly limited by the illustrative embodiments and examples set forth herein and that such examples and embodiments are presented by way of example only with the scope of the disclosure intended to be limited only by the claims set forth here.

1. A cooling article comprising:
a thermally conductive layer; and
a heat absorbing layer comprising a phase change material having a phase change temperature greater than 0° C.

2. The cooling article of claim 1, wherein the phase change material is in a flowable form at temperatures above and below the phase change temperature.

3. (canceled)

4. The cooling article of claim 1, wherein the heat absorbing layer has a cooling capacity of 100 kJ or less when cooled to below the phase change temperature.

5. (canceled)

6. The cooling article of claim 1, wherein the heat absorbing layer comprises two or more phase change materials having different phase change temperatures, optionally wherein the heat absorbing layer comprises a first phase change material having a phase change temperature in a range of −12° C. to 5° C. and a second phase change material having a phase change temperature greater than the first phase change material and in a range of 5° C. to 15° C.

7. The cooling article of claim 1, wherein the heat absorbing layer comprises 200 g or less of the phase change material.

8. The cooling article of claim 1, wherein the phase change material is encapsulated.

9. The cooling article of claim 1, wherein the phase change material is embedded in a thermally conductive material comprising a gel, a grease, a paste, a porous ceramic, a porous mineral, or a soft elastomer.

10. (canceled)

11. (canceled)

12. The cooling article of claim 1, wherein the thermally conductive layer comprises stainless steel, thermally conductive polymer, metalized textile fiber, silver, silver alloy, copper, copper alloy, aluminum, aluminum alloy 3003, Al—Ni—La alloy, tinned copper filament, boron nitride, or a combination thereof.

13. (canceled)

14. (canceled)

15. The cooling article of claim 1 further comprising an inner layer having a contact surface constructed to be placed against a subject to be cooled, the inner layer comprising a thermally conductive material, optionally wherein the contact surface comprises a plurality of spacers or protrusions extending from an inner surface of the cooling article, the plurality of spacers comprising a thermally conductive material and optionally non-thermally conductive material, optionally wherein the plurality of spacers or protrusions comprise ridges, loops, or fibers forming a thermal velvet.

16-18. (canceled)

19. The cooling article of claim 1, wherein the phase change material is disposed within a packet, where in the packet may be selectively removed from the cooling article.

20-25. (canceled)

26. The cooling article of claim 1 further comprising:
a sleeve constructed to be applied to a limb, the sleeve comprising:
a dorsal region positionable adjacent a dorsal aspect of the limb;
a palmar/plantar region positionable adjacent a palmar/plantar aspect of the limb;
a medial region positionable adjacent a medial aspect of the limb;
a lateral region positionable adjacent a lateral aspect of the limb;
a heat absorbing region that is present in one or more of the palmar/plantar, medial, and lateral regions;
an inner layer forming a contact surface with the limb;
an outer layer; and
wherein the phase change material is disposed within the heat absorbing region between the inner layer and the outer layer, the phase change material being flowable at temperatures below and above the phase change temperature.

27-34. (canceled)

35. A cooling article comprising:
a passive cooling layer; and
reinforcing fibers configured to support the passive cooling layer,
wherein the cooling article is configured to conform to a portion of a subject's body.

36. The cooling article of claim 35 wherein the reinforcing fibers are woven into the passive cooling layer.

37. (canceled)

38. (canceled)

39. The cooling article of claim 35, further comprising an inner layer constructed to be placed against a subject to be cooled, optionally wherein the inner layer comprises a plurality of spacers extending from an inner surface of the cooling article, the plurality of spacers comprising a thermally conductive material, optionally wherein the plurality of spacers are formed of silicone, and optionally wherein the spacers are formed by extrusion or 3D printing, optionally wherein the plurality of spacers comprise ridges or loops.

40. The cooling article of claim 35, comprising an inner layer comprising a thermally conductive material, optionally wherein the thermally conductive material comprises stainless steel, thermally conductive polymer, metalized textile fiber, silver, silver alloy, copper, copper alloy, aluminum, aluminum alloy 3003, Al—Ni—La alloy, tinned copper filament, boron nitride, or a combination thereof, optionally wherein the thermally conductive material comprises a woven material.

41-51. (canceled)
